(19) United States
(12) Patent Application Publication
(10) Pub. No.: US 2025/0228832 A1
(43) Pub. Date: Jul. 17, 2025

(54) METHODS FOR TREATING DISORDERS AMELIORATED BY MUSCARINIC RECEPTOR ACTIVATION

(71) Applicant: KARUNA THERAPEUTICS, INC., Boston, MA (US)

(72) Inventors: Steven PAUL, Boston, MA (US); Christian FELDER, Boston, MA (US); James Allen MONN, Boston, MA (US); Michael POPIOLEK, Boston, MA (US); Samantha YOHN, Derry, NH (US)

(21) Appl. No.: 18/853,947

(22) PCT Filed: Apr. 7, 2023
(86) PCT No.: PCT/US2023/065498

§ 371 Date: Oct. 3, 2024

Related U.S. Application Data
(60) Provisional application No. 63/365,062, filed on May 20, 2022.
(60) Provisional application No. 63/362,629, filed on Apr. 7, 2022.

(51) Int. Cl. A61K 31/4439; A61K 31/221; A61K 31/407; A61K 31/439; A61K 47/02; A61K 47/26; A61K 47/38; A61P 25/18

(52) U.S. Cl. CPC A61K 31/4439; A61K 31/221; A61K 31/407; A61K 31/439; A61K 47/02; A61K 47/26; A61K 47/38; A61P 25/18

(57) ABSTRACT

Provided herein is a method for treating a disorder ameliorated by muscarinic receptor activation in a patient in need thereof, comprising administering to the patient a muscarinic orthosteric agonist, such as xanomeline and/or a salt thereof and an M1 or M4 muscarinic positive allosteric modulator (PAM). Related pharmaceutical compositions are also provided.

This application claims the benefit of priority of U.S. Provisional Patent Application Ser. No. 63/362,629 filed Apr. 7, 2022, and also claims the benefit of priority of the U.S. Provisional Patent Application Ser. No. 63/365,062 filed May 20, 2022, the disclosures of which are each incorporated by reference in their entireties for all purposes.

The present disclosure relates to compositions and their application as pharmaceuticals for treating disorders ameliorated by activating muscarinic receptors in a human or animal subject.

Activating the muscarinic system through muscarinic agonists may treat several diseases, such as dementia-related psychosis, schizophrenia, Alzheimer's disease, Parkinson's disease, depression, movement disorders, drug addiction, pain, and neurodegeneration, such as tauopathies or synucleinopathies.

In a double-blind placebo-controlled trial of schizophrenic patients using xanomeline (25-75 mg TID, 75-225 mg TDD), an orthosteric muscarinic cholinergic receptor agonist with preferential activity at the M1 and M4 subtype receptors, positive (psychosis), negative, and cognitive symptoms of schizophrenia were improved. However, because xanomeline is also bound to muscarinic receptors outside the brain, it has many serious side effects, including GI side effects, cardiac side effects, and hypersalivation. Dose-limited adverse events were problematic and led to high discontinuation rates (including a 56% dropout rate in a 26-week study of Alzheimer's disease) and eventually to discontinuation of xanomeline development.

There remains a need in the art for a pharmaceutical composition of a muscarinic agonist, such as xanomeline, with increased tolerability, in order to treat cognitive and psychotic disorders. The following embodiments and aspects thereof are described and illustrated with compositions and methods, which are meant to be exemplary and illustrative, not limiting in scope. In various embodiments, one or more of the above-described problems have been reduced or eliminated, while other embodiments are directed to other improvements.

### SUMMARY

Provided herein is a method for treating a disorder ameliorated by muscarinic receptor activation in a patient in need thereof, comprising administering to the patient a muscarinic orthosteric agonist, such as xanomeline and/or a salt thereof, and a muscarinic positive allosteric modulator, wherein the muscarinic orthosteric agonist and the muscarinic positive allosteric modulator operate on the same muscarinic subtype. In certain embodiments, the method further comprises administering to the patient a muscarinic antagonist, such as trospium chloride.

Also provided herein is a method for treating a disorder ameliorated by muscarinic receptor activation in a patient in need thereof, comprising administering to the patient M1 or M4 a muscarinic orthosteric agonist, such as xanomeline and/or a salt thereof, and an M1 or M4 muscarinic positive allosteric modulator (PAM). In certain embodiments, the method further comprises administering to the patient a muscarinic antagonist, such as trospium chloride.

Further provided herein is a pharmaceutical composition, comprising an M1 or M4 a muscarinic orthosteric agonist, such as xanomeline and/or a salt thereof, an M1 or M4 muscarinic positive allosteric modulator (PAM), and at least one pharmaceutically acceptable excipient. In certain embodiments, the pharmaceutical composition further comprises a muscarinic antagonist, such as trospium chloride.

Further aspects and advantages will be apparent to those of ordinary skill in the art from a review of the following detailed description. While the dosage form, method of making, and treatment method are susceptible to embodiments in various forms, the description hereafter includes specific embodiments to understand that the disclosure is illustrative and is not intended to limit the disclosure to the specific embodiments described herein.

### BRIEF DESCRIPTION OF THE DRAWINGS

The disclosure will be readily understood by the following detailed description in conjunction with the accompanying drawings, wherein like reference numerals designate like structural elements. The drawings provide exemplary embodiments or aspects of the disclosure and do not limit the scope of the disclosure.

FIG. 1 shows the in vitro cooperativity of MK-4710 with acetylcholine at the M4 mAChR.

FIG. 2 shows the in vitro cooperativity of MK-4710 with xanomeline at the M4 mAChR.

### DETAILED DESCRIPTION

Provided is a method for treating a disorder ameliorated by muscarinic receptor activation in a patient in need thereof, comprising administering to the patient a muscarinic orthosteric agonist, such as xanomeline and/or a salt thereof, and an M1 or M4 muscarinic positive allosteric modulator (PAM). In certain embodiments, the method further comprises administering to the patient a muscarinic antagonist, such as trospium chloride.

### Methods of Treating

Contrary to the prior art, administration of xanomeline and/or a salt thereof with one or more positive allosteric modulators (PAMs) displays a new cooperative effect, which unexpectedly increases the efficacy of the xanomeline (an efficacy shift, referred to as beta, Q) and/or a salt thereof. In-vitro results suggest that co-administration of M4 PAMs increase the efficacy of synthetic muscarinic agonists (an efficacy shift, referred to as beta, β) at the M4 receptor at concentrations between 3.33×10⁻⁶ and 10⁻⁸ M. Agonist pharmacology (referred to as tau, τ) was also observed when agonists such as xanomeline were co-treated with an M4 PAM. Native muscarinic acetylcholine receptor ligand (acetylcholine) potency can also be increased, where M4 PAMs predominantly impacts acetylcholine potency (through an affinity shift, referred to as alpha, α) with modest impact on the efficacy shift, (referred to as beta, β) and no impact on pharmacology (referred to as tau, τ). The M4 muscarinic acetylcholine receptor can be selectively and preferentially activated when muscarinic agonists are combined with an M4 PAM to obtain the desired effects produced by embodiments of the invention. Further, synthetic muscarinic agonists when combined with an M4 PAM are able to obtain a novel effect relative to acetylcholine (efficacy shift f compared to affinity shift α), with lower doses than if the synthetic muscarinic agonist was administered alone. One or more alpha, beta, and tau properties could also be found in a single M4 PAM in combination with an orthosteric agonist such as xanomeline.

An affinity shift refers to a change in the concentration or dose of a drug to achieve a particular level of biological effect. In other words, an affinity shift occurs when a drug's affinity for its target is altered, resulting in a change in its potency without changing the maximal effect it can produce. In combination therapy, an affinity shift may result in a lower dose of one or both drugs to achieve the desired therapeutic effect.

For example, in embodiments with an affinity shift, the timing of administration of the two drugs may affect the level of receptor occupancy and the extent of allosteric modulation. If the two drugs are administered simultaneously, the affinity shift may be more pronounced than when the drugs are administered sequentially. On the other hand, if the drugs are administered sequentially, the order in which they are given may also affect the affinity shift.

An efficacy shift, on the other hand, refers to a change in the maximal biological effect that a drug can produce. In other words, an efficacy shift occurs when a drug's ability to activate its target is altered, resulting in a change in the maximal effect it can produce without changing its potency. In combination therapy, an efficacy shift may result in a greater therapeutic effect than either drug alone but may also require a higher overall dose of both drugs to achieve this effect.

In certain embodiments with an efficacy shift, the timing of administration may affect the extent of receptor desensitization and resensitization. For example, if the drugs are administered simultaneously, the efficacy shift may be more pronounced than when the drugs are administered sequentially. The timing and order of administration of the two drugs may also affect the onset and duration of the efficacy shift.

A tau shift refers to a change in how a receptor responds to a drug, such as switching from agonism to allosteric modulation. Tau shifts can have positive or negative effects on therapeutic efficacy depending on the therapeutic outcome and the mechanism of action of the drug. In certain embodiments, a tau shift may be desirable as it can result in greater therapeutic efficacy or improved safety profile. For example, a tau shift from agonism to allosteric modulation may reduce the risk of adverse effects associated with high levels of receptor activation while still allowing for therapeutic efficacy. In other embodiments, a tau shift may be undesirable if it results in a loss of efficacy or increased adverse effects. In certain embodiments, a tau shift may indicate a loss of selectivity, which can reduce the therapeutic window of a drug. In certain embodiments, combining a muscarinic orthosteric agonist and a PAM does not provide a tau shift.

In certain embodiments, the muscarinic orthosteric agonist is acetylcholine and the PAM is an M4 PAM, and wherein the M4 PAM cooperates with acetylcholine through an affinity shift.

In certain embodiments, the muscarinic orthosteric agonist is xanomeline and the PAM is an M4 PAM, and wherein the M4 PAM cooperates with xanomeline through an efficacy shift.

In certain embodiments, the muscarinic orthosteric agonist is cevimeline and the PAM is an M4 PAM, and wherein the M4 PAM cooperates with cevimeline through an efficacy shift.

In certain embodiments, the muscarinic orthosteric agonist is 3-(1-methyl-1,4,5,6-tetrahydropyridin-2-yl)-4-(pentyloxy)-1,2,5-thiadiazole (C₅-xanomeline analog) and the PAM is an M4 PAM, and wherein the M4 PAM cooperates with C₅-xanomeline analog through an efficacy shift.

In certain embodiments, the muscarinic orthosteric agonist is 3-(heptyloxy)-4-(1-methyl-1,4,5,6-tetrahydropyridin-2-yl)-1,2,5-thiadiazole (C₇-xanomeline analog) and the PAM is an M4 PAM, and wherein the M4 PAM cooperates with C₇-xanomeline analog through an efficacy shift.

While activators of M1 and M4 muscarinic receptors have been suggested to be efficacious treatments for schizophrenia, the activation of muscarinic receptors located outside the brain has resulted in side effects that prevented the successful development of xanomeline. For instance, in both Phase I and subsequent trials, the muscarinic agonist xanomeline had unacceptable GI and other side effects linked to the binding of muscarinic receptors in peripheral tissues, or tissues outside the brain. The most common adverse events observed with administering xanomeline were nausea, vomiting, diarrhea, excessive sweating, and excessive salivation (so-called cholinergic parasympathetic adverse events).

The term “activator” means a molecule described as an agonist, orthosteric agonist, partial agonist, co-agonist, physiological agonist, potentiator, stimulator, allosteric potentiator, positive allosteric modulator, allosteric agonist, or a molecule that increases the activity or signaling of receptors directly or indirectly. In certain embodiments, the actives are activators chosen from orthosteric agonists and positive allosteric modulators.

“Muscarinic orthosteric agonists,” “orthosteric muscarinic agonists,” or “muscarinic agonists” refers to agents that activate the muscarinic acetylcholine receptor. “Orthosteric” refers to a site of a receptor in which the endogenous ligand, acetylcholine, binds to produce its effect. That is, muscarinic orthosteric agonists bind to the site of the muscarinic receptor in which endogenous muscarinic ligands bind to produce their effects.

There are five different muscarinic receptors labeled M1-M5. Muscarinic cholinergic receptors are G-protein coupled receptors with five different receptor subtypes (M1-M5), each of which is found in the CNS with different tissue distributions. The term “muscarinic receptors” refers to G-protein-linked receptors that bind the neurotransmitter acetylcholine.

To date, five subtypes of the muscarinic receptor have been identified. “M1” means the subtype one muscarinic receptor. “M2” means the subtype two muscarinic receptor. “M3” means the subtype three muscarinic receptor. “M4” means the subtype four muscarinic receptor. “M5” means the subtype five muscarinic receptor.

A muscarinic agonist may be selective or prefer binding to only one muscarinic receptor subtype, partially selective or prefer binding to two to four subtypes, or non-selective, preferring to bind to each of the five subtypes. Muscarinic agonists are parasympathomimetic. Their mechanism of action is different depending on which receptor is activated. For instance, mood stabilizers lithium and valproic acid, used for treating bipolar depression, may affect the muscarinic system, mainly through the M4 subtype receptor.

Genetic evidence directly links the muscarinic system and alcohol addiction. Examples of orthosteric muscarinic agonists are shown on Table 1.

TABLE 1
Examples of orthosteric muscarinic agonists
with observed preference mAChR subtypes

| | Muscarinic Receptor Subtype | | | | |
|---|---|---|---|---|---|
| Agonist | M1 | M2 | M3 | M4 | M5 |
| 77-LH-28-1 | X | | | | |
| A 72055 | X | X | | | |
| AF 125 | X | | | | |
| AF 150(S) | X | | | | |
| Aceclidine | | | X | | |
| Alvameline | X | | | | |
| Arecoline | X | X | X | | |
| Bethanechol | X | X | X | X | X |
| Carbachol | X | X | X | X | X |
| Cevimeline | X | | X | | |
| CI 1017 | X | | | X | |
| CMI 1145 | | X | | X | |
| CMI 936 | | | | X | |
| FPL 14995 | X | | | | |
| Furmethide | | X | | X | |
| HTL-0016878 | | | | X | |
| Iperoxo | X | X | X | X | X |
| Itrameline | X | | X | | |
| KST 5452 | X | | | | |
| L 670,548 | | | X | | |
| L 687,306 | X | | | | |
| L 689,660 | X | | X | | |
| L 686,986 | X | | | | |
| Methacholine | X | X | X | X | X |
| N-desmethylclozapine | X | | | | |
| MCD 386 | X | | | | |
| Milameline | X | X | X | X | X |
| NC 111585 | X | | | | |
| Nebracetam | X | | | | |
| NGX267 | X | | | | |
| ORG 20091 | X | | | | |
| Oxotremorine | X | X | X | X | X |
| PD 142505 | X | | | | |
| PD 151832 | X | | | | |
| PDC 008004 | | X | | | |
| Pilocarpine | X | X | X | X | |
| RU 35963 | | | X | | |
| Sabcomeline | X | | | | |
| SR 46559A | X | | | | |
| Talsaclidine | X | | | | |
| Tazomeline | X | X | X | X | X |
| Thiopilocarpine | X | | | | |
| Tremorine | X | X | X | X | X |
| Vedaclidine | X | | | X | |
| Xanomeline | X | | | X | |
| WAY-131256 | X | X | | | |
| WAY-132983 | X | | | | |
| YM 796 | X | | | | |
| YM 954 | X | | | | |

Examples of orthosteric muscarinic agonists include, but are not limited to, 77-LH-28-1, A 72055, AF 125, AF 150(S), aceclidine, alvameline, arecoline, bethanechol, carbachol, cevimeline, CI 1017, CMI 1145, CMI 936, FPL 14995, furmethide, HTL-0016878, iperoxo, itrameline, KST 5452, L 670,548, L 687,306, L 689,660, L 686,986, methacholine, N-desmethylclozapine, MCD 386, milameline, NC 111585, nebracetam, NGX267, ORG 20091, oxotremorine, PD 142505, PD 151832, PDC 008004, pilocarpine, RU 35963, sabcomeline, SR 46559A, talsaclidine, tazomeline, thiopilocarpine, tremorine, vedaclidine, xanomeline, WAY-131256, WAY-132983, YM 796, and YM 954. In certain embodiments, the orthosteric muscarinic agonist is xanomeline, which activates both M1 and M4 muscarinic receptors.

Examples of M1 orthosteric muscarinic agonists include, but are not limited to, 77-LH-28-1, A 72055, AF 125, AF 150(S), alvameline, bethanechol, carbachol, cevimeline, CI 1017, FPL 14995, iperoxo, itrameline, KST 5452, L 687,306, L 689,660, L 686,986, methacholine, N-desmethylclozapine, MCD 386, milameline, NC 111585, nebracetam, NGX267, ORG 20091, oxotremorine, PD 142505, PD 151832, pilocarpine, sabcomeline, SR 46559A, talsaclidine, tazomeline, thiopilocarpine, tremorine, vedaclidine, xanomeline, WAY-131256, WAY-132983, YM 796, and YM 954. In certain embodiments, the M1 orthosteric muscarinic agonist is xanomeline.

Examples of M2 orthosteric muscarinic agonists include, but are not limited to, A 72055, bethanechol, carbachol, CMI 1145, furmethide, iperoxo, methacholine, milameline, oxotremorine, PDC 008004, pilocarpine, tazomeline, tremorine, and WAY-131256.

Examples of M3 orthosteric muscarinic agonists include, but are not limited to, aceclidine, arecoline, bethanechol, carbachol, cevimeline, iperoxo, itrameline, L 670,548, L 689,660, methacholine, milameline, oxotremorine, pilocarpine, RU 35963, tazomeline, and tremorine.

Examples of M4 orthosteric muscarinic agonists include, but are not limited to, bethanechol, carbachol, CI 1017, CMI 1145, CMI 936, furmethide, HTL-0016878, iperoxo, methacholine, milameline, oxotremorine, pilocarpine, tazomeline, tremorine, vedaclidine, and xanomeline. In certain embodiments, the M4 orthosteric muscarinic agonist is xanomeline.

Examples of M5 orthosteric muscarinic agonists include, but are not limited to, bethanechol, carbachol, iperoxo, methacholine, milameline, tazomeline, and tremorine. [0036]77-LH-28-1 is 1-(3-(4-butyl-1-piperidinyl)propyl)-3,3-dihydro-2(1H)-quinolinone. L 670,578 is 1-azabicyclo[2.2.1]heptane,3-(3-methyl-1,2,4-oxadiazol-5-yl)-monohydrochloride. L-689,660 is 1-azabicyclo[2.2.2]octane,3-(6-chloropyrazinyl)maleate. N-desmethylclozapine is also called norclosapine or NDMC. CI-1017 is also known as PD 141606. WAY-131256 is 3S,4R-azabicyclo[2.2.1]heptan-3-methylcarbamate.

Cevimeline (Evoxac™) is a synthetic analog of the natural alkaloid muscarine with a particular agonistic effect on M1 and M3 receptors. It is used to treat dry mouth (xerostomia) and Sjögren's syndrome.

In certain embodiments, the PAM is an M1 PAM chosen from BQCA, GSK 1034702, M1-PAM-A, M1-PAM-B, MK-7622, PF-6767832, PQCA, TAK-071, VU319, VU0119498, VU0453595, VU0456940, VU0486846, VU0550164, VU6004256, VU6005877, and VU6007477. Structures and IUPAC names for examples of M1 PAMs are shown in Table 2.

TABLE 2
Examples of M1 PAMs

| Structure | Name |
|---|---|
| 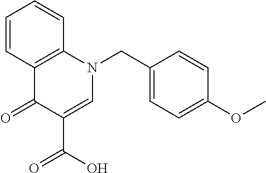 [SMILES: COc1ccc(Cn2cc(C(=O)O)c(=O)c3ccccc32)cc1] | BQCA; 1-(4-methoxybenzyl)-4-oxo- 1,4-dihydroquinoline-3-carboxylic acid |
| 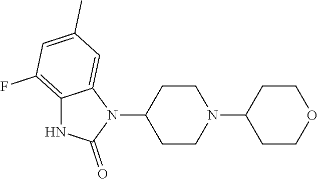 [SMILES: Cc1cc(F)c2[nH]c(=O)n(C3CCN(C4CCOCC4)CC3)c2c1] | GSK 1034702; 4-fluoro-6-methyl-1- (1-(tetrahydro-2H-pyran-4-yl)-4- piperidinyl)-1,3-dihydro-2H- benzimidazol-2-one |
| 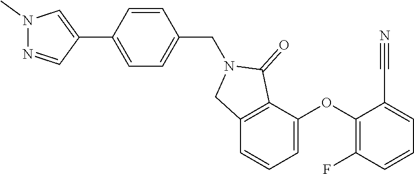 [SMILES: Cn1cc(-c2ccc(CN3Cc4cccc(Oc5c(F)cccc5C#N)c4C3=O)cc2)cn1] | M1-PAM-A; 3-fluoro-2-((2-(4-(1- methyl-1H-pyrazol-4-yl)benzyl)-3- oxo-2,3-dihydro-1H-isoindol-4- yl)oxy)benzonitrile |
| 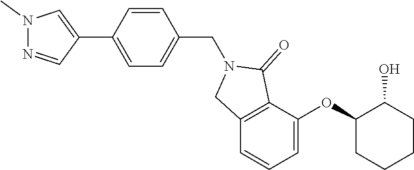 [SMILES: Cn1cc(-c2ccc(CN3Cc4cccc(O[C@@H]5CCCC[C@H]5O)c4C3=O)cc2)cn1] | M1-PAM-B; 7-(((1S,2S)-2- hydroxycyclohexyl)oxy)-2-(4-(1- methyl-1H-pyrazol-3- yl)benzyl)isoindolin-1-one |
| 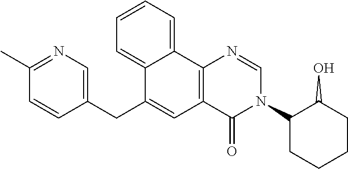 | MK-7622; 3-((1S,2R)-2- hydroxycyclohexyl)-6-((6- methylpyridin-3-yl)methyl)benzo [h]quinazolin-4(3H)-one |
| 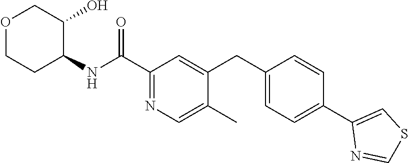 [SMILES: Cc1cnc(C(=O)N[C@H]2CCOC[C@@H]2O)cc1Cc1ccc(-c2cscn2)cc1] | PF-6767832; N-((3R,4S)-3- hydroxytetrahydro-2H-pyran-4-yl)-5- methyl-4-[4-(1,3-thiazol-4- yl)benzyl]pyridine-2-carboxamide |
| 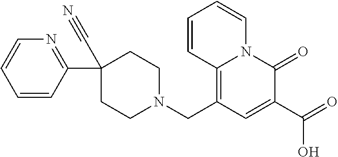 [SMILES: N#CC1(c2ccccn2)CCN(Cc2cc(C(=O)O)c(=O)n3ccccc23)CC1] | PQCA; 1-((4-cyano-4-(pyridin-2- yl)piperidin-1-yl)methyl)-4-oxo-4H- quinolizine-3-carboxylic acid |
| 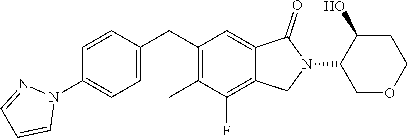 | TAK-071; 6-(4-(1H-pyrazol-1- yl)benzyl)-4-fluoro-2-((3S,4S)-4- hydroxytetrahydro-2H-pyran-3-yl)-5- methylisoindolin-1-one |
| 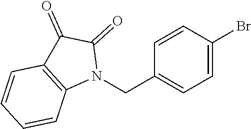 [SMILES: O=C1C(=O)N(Cc2ccc(Br)cc2)c2ccccc21] | VU0119498; 1-[(4- bromophenyl)methyl]indole-2,3-dione |
| 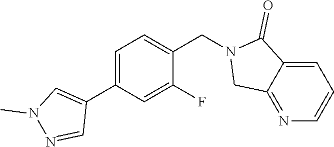 [SMILES: Cn1cc(-c2ccc(CN3Cc4ncccc4C3=O)c(F)c2)cn1] | VU0453595; 6-[[2-Fluoro-4-(1- methyl-1H-pyrazol-4- yl)phenyl]methyl]-6,7-dihydro-5H- pyrrolo[3,4-b]pyridin-5-one |
| 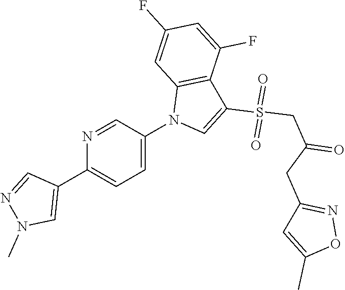 [SMILES: Cc1cc(CC(=O)CS(=O)(=O)c2cn(-c3ccc(-c4cnn(C)c4)nc3)c3cc(F)cc(F)c23)no1] | VU0456940; 1-[4,6-difluoro-1-[6-(1- methylpyrazol-4-yl)pyridin-3- yl]indol-3-yl]sulfonyl-3-(5-methyl- 1,2-oxazol-3-yl)propan-2-one |
| 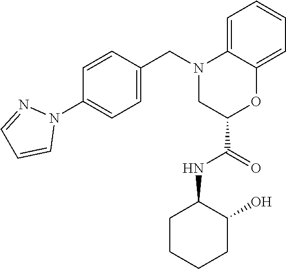 [SMILES: O=C(N[C@@H]1CCCC[C@H]1O)[C@@H]1CN(Cc2ccc(-n3cccn3)cc2)c2ccccc2O1] | VU0486846; (R)-4-(4-(1H-pyrazol-1- yl)benzyl)-N-((1S,2S)-2- hydroxycyclohexyl)-3,4-dihydro-2H- benzo[b][1,4]oxazine-2-carboxamide |
| 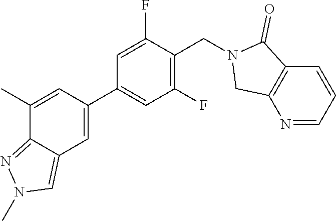 [SMILES: Cc1cc(-c2cc(F)c(CN3Cc4ncccc4C3=O)c(F)c2)cc2cn(C)nc12] | VU0550164; 6-(4-(2,7-dimethyl-2H- indazol-5-yl)-2,6-difluorobenzyl)-6,7- dihydro-5H-pyrrolo[3,4-b]pyridin-5- one |
| 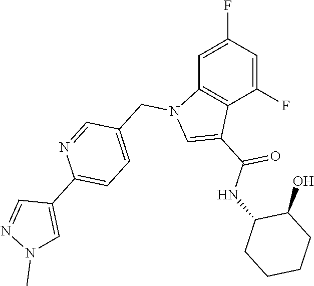 [SMILES: Cn1cc(-c2ccc(Cn3cc(C(=O)N[C@H]4CCCC[C@@H]4O)c4c(F)cc(F)cc43)cn2)cn1] | VU6004256; 4,6-difluoro-N-[(1S,2S)- 2-hydroxycyclohexyl]-1-[6-(1- methyl-1H-pyrazol-4-yl)-3- pyridinyl]methyl]-1H-indole-3- carboxamide |
| 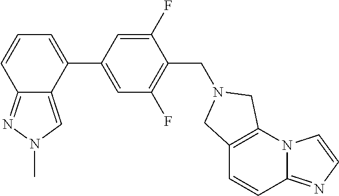 [SMILES: Cn1cc2c(-c3cc(F)c(CN4Cc5ccc6nccn6c5C4)c(F)c3)cccc2n1] | VU6005877; 2-(2,6-difluoro-4-(2- methyl-2H-indazol-4-yl)benzyl)-2,3- dihydro-1H-imidazo[1,2- a]pyrrolo[3,4-e]pyridin-1-one |
| 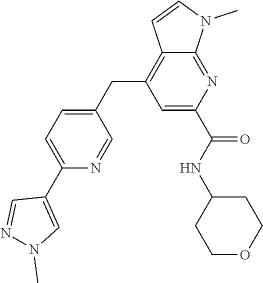 [SMILES: Cn1cc(-c2ccc(Cc3cc(C(=O)NC4CCOCC4)nc4c3ccn4C)cn2)cn1] | VU6007477; 1-methyl-4-((6-(1- methyl-1H-pyrazol-4-yl)pyridin-3- yl)methyl)-N-(tetrahydro-2H-pyran-4- yl)-1H-pyrrolo[2,3-b]pyridine-6- carboxamide |

In certain embodiments, the PAM is an M4 PAM chosen from emraclidine, LY2033298, LY2119620, VU0152099, VU0152100, VU0467154, VU0467485, VU0473619, VU0476406, VU6000918, VU6002703, VU6009003, VU6009453, 2-[14-[(1,3-dihydro-5-isobenzofuranyl)oxy]-1-piperidinyl]-6,7-dihydro-3-methyl-5H-pyrrolo[3,4-b]pyridin-5-one, and 6-(2-methyl-3-oxoisoindolin-5-yl)-5-(1-((1-methylcyclopentyl)methyl)-1H-pyrazol-4-yl)picolinonitrile. Structures and IUPAC names for M4 PAMs are shown on Table 3.

TABLE 3
Examples of M4 PAMs

| Structure | Name |
|---|---|
| 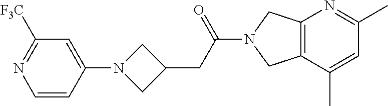 [SMILES: Cc1cc(C)c2c(n1)CN(C(=O)CC1CN(c3ccnc(C(F)(F)F)c3)C1)C2] | Emraclidine |
| 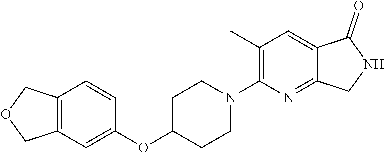 [SMILES: Cc1cc2c(nc1N1CCC(Oc3ccc4c(c3)COC4)CC1)CNC2=O] | MK-4710; 2-[4-[(1,3-dihydro-5- isobenzofuranyl)oxy]-1-piperidinyl]-6,7- dihydro-3-methyl-5H-pyrrolo[3,4-b]pyridin- 5-one |
| 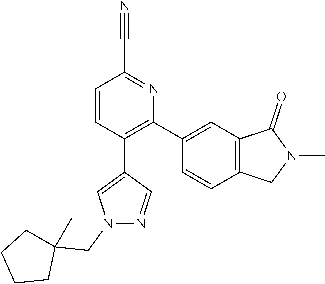 [SMILES: CN1Cc2ccc(-c3nc(C#N)ccc3-c3cnn(CC4(C)CCCC4)c3)cc2C1=O] | 6-(2-methyl-3-oxoisoindolin-5-yl)-5-(1-((1- methylcyclopentyl)methyl)-1H-pyrazol-4- yl)picolinonitrile |
| 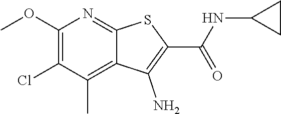 [SMILES: COc1nc2sc(C(=O)NC3CC3)c(N)c2c(C)c1Cl] | LY2033298; 3-amino-5-chloro-N- cyclopropyl-6-methoxy-4-methyl-thieno[2,3- b]pyridine-2-carboxamide |
| 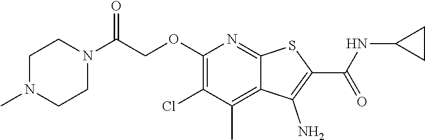 [SMILES: Cc1c(Cl)c(OCC(=O)N2CCN(C)CC2)nc2sc(C(=O)NC3CC3)c(N)c12] | LY2119620; 3-amino-5-chloro-N- cyclopropyl-4-methyl-6-(2-(4- methylpiperazin-1-yl)-2- oxoethoxy)thieno[2,3-b]pyridine-2- carboxamide |
| 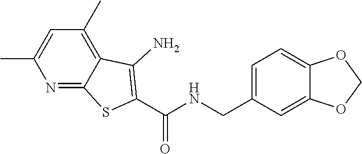 [SMILES: Cc1cc(C)c2c(N)c(C(=O)NCc3ccc4c(c3)OCO4)sc2n1] | VU0152099; 3-amino-N-(1,3-benzodioxol- 5-ylmethyl)-4,6-dimethylthieno[5,4- b]pyridine-2-carboxamide |
| 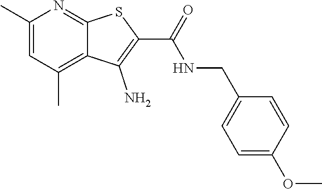 | VU0152100; 3-amino-N-[(4- methoxyphenyl)methyl]-4,6-dimethyl- thieno[2,3-b]pyridine-2-carboxamide |
| 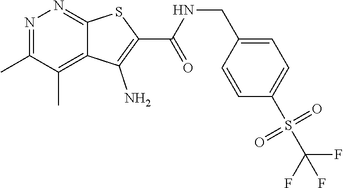 [SMILES: Cc1nnc2sc(C(=O)NCc3ccc(S(=O)(=O)C(F)(F)F)cc3)c(N)c2c1C] | VU0467154; 5-amino-3,4-dimethyl-N-(4- ((trifluoromethyl)sulfonyl)benzyl)thieno[2,3- c]pyridazine-6-carboxamide |
| 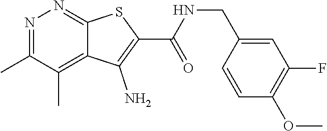 [SMILES: COc1ccc(CNC(=O)c2sc3nnc(C)c(C)c3c2N)cc1F] | VU0467485; 5-amino-N-(3-fluoro-4- methoxybenzyl)-3,4-dimethylthieno[2,3- c]pyridazine-6-carboxamide |
| 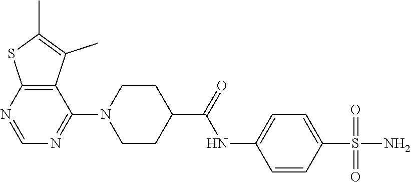 [SMILES: Cc1sc2ncnc(N3CCC(C(=O)Nc4ccc(S(N)(=O)=O)cc4)CC3)c2c1C] | VU0473619; 1-(5,6-dimethylthieno[2,3- d]pyrimidin-4-yl)-N-(4- sulfamoylphenyl)piperidine-4-carboxamide |
| 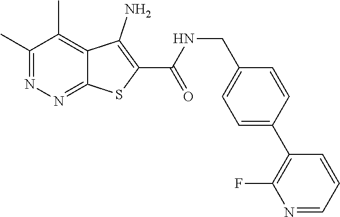 [SMILES: Cc1nnc2sc(C(=O)NCc3ccc(-c4cccnc4F)cc3)c(N)c2c1C] | VU0476406; 5-amino-N-(4-(2-fluoropyridin- 3-yl)benzyl)-3,4-dimethylthieno[2,3- c]pyridazine-6-carboxamide |
| 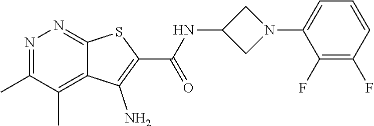 [SMILES: Cc1nnc2sc(C(=O)NC3CN(c4cccc(F)c4F)C3)c(N)c2c1C] | VU6000918; 5-amino-N-[1-(2, 3- difluorophenyl)azetidin-3-yl]-3, 4- dimethylthieno[2,3-c]pyridazine-6- carboxamide |
| 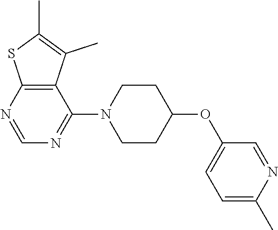 [SMILES: Cc1ccc(OC2CCN(c3ncnc4sc(C)c(C)c34)CC2)cn1] | VU6002703; 5,6-dimethyl-4-(4-((6- methylpyridin-3-yl)oxy)piperidin-1- yl)thieno[2,3-d]pyrimidine |
| 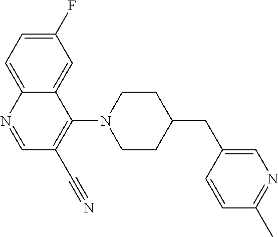 [SMILES: Cc1ccc(CC2CCN(c3c(C#N)cnc4ccc(F)cc34)CC2)cn1] | VU6009003; 6-fluoro-4-(4-((6- methylpyridin-3-yl)methyl)piperidin-1- yl)quinoline-3-carbonitrile |
| 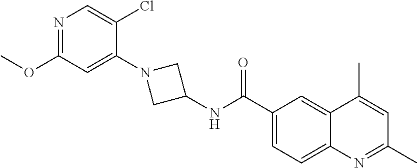 | VU6009453; N-(1-(5-chloro-2- methoxypyridin-4-yl)azetidin-3-yl)-2,4- dimethylquinoline-6-carboxamide |

Emraclidine, or Cerevel-231, is a selective M4 PAM.

MK-4710, or Merck clinical M4 PAM, is a selective M4 PAM.

In certain embodiments, the method further comprises administering an anticholinergic agent. In certain embodiments, the anticholinergic agent is a muscarinic antagonist. In certain embodiments, the muscarinic antagonist is chosen from trospium, tolterodine, darifenacin, solifenacin, fesoterodine, scopolamine, N-methylscopolamine, and a salt thereof.

In certain embodiments, the muscarinic antagonist is a salt of trospium. In certain embodiments, the salt of trospium is chosen from trospium chloride, trospium bromide, trospium iodide, and trospium saccharinate. In certain embodiments, the muscarinic antagonist is trospium chloride.

In certain embodiments, the muscarinic antagonist and the orthosteric muscarinic agonist are administered in a first composition and the PAM is administered in a second composition.

Before co-administering the disclosed combinations, patients may have a lead-in period from one to 28 days, during which lead-in period trospium chloride and/or the PAM is given alone. In one embodiment, the trospium chloride and/or PAM are co-administered for one or more dose periods before co-administering xanomeline to accumulate trospium chloride and/or PAM in the body or for the trospium chloride and/or PAM to reach or approach steady-state exposure levels. This accumulation or higher exposure levels of the trospium chloride increases the blockade of muscarinic receptors outside of the brain and reduces adverse events when xanomeline is administered. Likewise, this accumulation or higher exposure levels to the PAM simplifies the process for titrating the amount of xanomeline and/or a salt thereof. In another embodiment, the trospium chloride and/or PAM are co-administered for one or more days before xanomeline.

The present disclosure provides a method for treating a disorder ameliorated by muscarinic receptor activation in a patient in need thereof, comprising administering to the patient a muscarinic orthosteric agonist and a muscarinic positive allosteric modulator (PAM), wherein the muscarinic orthosteric agonist and the PAM operate on the same muscarinic subtype. In certain embodiments, method further comprises administering to the patient a muscarinic antagonist, such as trospium chloride.

In certain embodiments, the subtype is M1, and the muscarinic orthosteric agonist is chosen from 77-LH-28-1, A 72055, AF 125, AF 150(S), alvameline, bethanechol, carbachol, cevimeline, CI 1017, FPL 14995, iperoxo, itrameline, KST 5452, L 687,306, L 689,660, L 686,986, methacholine, N-desmethylclozapine, MCD 386, milameline, NC 111585, nebracetam, NGX267, ORG 20091, oxotremorine, PD 142505, PD 151832, pilocarpine, sabcomeline, SR 46559A, talsaclidine, tazomeline, thiopilocarpine, tremorine, vedaclidine, xanomeline, WAY-131256, WAY-132983, YM 796, and YM 954, and the PAM is chosen from BQCA, GSK 1034702, M1-PAM-A, M1-PAM-B, MK-7622, PF-6767832, PQCA, TAK-071, VU319, VU0119498, VU0453595, VU0456940, VU0486846, VU0550164, VU6004256, VU6005877, and VU6007477. In certain embodiments, method further comprises administering to the patient a muscarinic antagonist, such as trospium chloride.

In certain embodiments, the subtype is M1, the muscarinic orthosteric agonist is xanomeline, and the PAM is chosen from BQCA, GSK 1034702, M1-PAM-A, M1-PAM-B, MK-7622, PF-6767832, PQCA, TAK-071, VU319, VU0119498, VU0453595, VU0456940, VU0486846, VU0550164, VU6004256, VU6005877, and VU6007477. In certain embodiments, method further comprises administering to the patient a muscarinic antagonist, such as trospium chloride.

In certain embodiments, the subtype is M4, the muscarinic orthosteric agonist is chosen from bethanechol, carbachol, CI 1017, CMI 1145, CMI 936, furmethide, HTL-0016878, iperoxo, methacholine, milameline, oxotremorine, pilocarpine, tazomeline, tremorine, vedaclidine, and xanomeline, and the PAM is chosen from emraclidine, LY2033298, LY2119620, VU0152099, VU0152100, VU0467154, VU0467485, VU0473619, VU0476406, VU6000918, VU6002703, VU6009003, VU6009453, 2-[4-[(1,3-dihydro-5-isobenzofuranyl)oxy]-1-piperidinyl]-6,7-dihydro-3-methyl-5H-pyrrolo[3,4-b]pyridin-5-one, and 6-(2-methyl-3-oxoisoindolin-5-yl)-5-(1-((1-methylcyclopentyl)methyl)-1H-pyrazol-4-yl)picolinonitrile. In certain embodiments, method further comprises administering to the patient a muscarinic antagonist, such as trospium chloride.

In certain embodiments, the subtype is M4, the muscarinic orthosteric agonist is xanomeline, and the PAM is chosen from emraclidine, LY2033298, LY2119620, VU0152099, VU0152100, VU0467154, VU0467485, VU0473619, VU0476406, VU6000918, VU6002703, VU6009003, VU6009453, 2-[4-[(1,3-dihydro-5-isobenzofuranyl)oxy]-1-piperidinyl]-6,7-dihydro-3-methyl-5H-pyrrolo[3,4-b]pyridin-5-one, and 6-(2-methyl-3-oxoisoindolin-5-yl)-5-(1-((1-methylcyclopentyl)methyl)-1H-pyrazol-4-yl)picolinonitrile. In certain embodiments, method further comprises administering to the patient a muscarinic antagonist, such as trospium chloride.

In certain embodiments, the subtype is M4, the muscarinic orthosteric agonist is xanomeline, and the PAM is chosen from emraclidine, 2-[4-[(1,3-dihydro-5-isobenzofuranyl)oxy]-1-piperidinyl]-6,7-dihydro-3-methyl-5H-pyrrolo[3,4-b]pyridin-5-one, and 6-(2-methyl-3-oxoisoindolin-5-yl)-5-(1-((1-methylcyclopentyl)methyl)-1H-pyrazol-4-yl)picolinonitrile. In certain embodiments, method further comprises administering to the patient a muscarinic antagonist, such as trospium chloride.

In certain embodiments, the dosage of the muscarinic orthosteric agonist ranges between 5 mg and 700 mg muscarinic orthosteric agonist per day, such as between 25 mg and 300 mg muscarinic orthosteric agonist, such between 25 mg and 30 mg, 30 mg and 35 mg, 35 mg and 40 mg, 40 mg and 45 mg, 45 mg and 50 mg, 50 mg and 55 mg, 55 mg and 60 mg, 60 mg and 65 mg, 65 mg and 70 mg, 70 mg and 75 mg, 75 and 80 mg, 80 mg and 85 mg, 85 mg and 90 mg, 90 mg and 95 mg, 95 mg and 100 mg, 105 mg and 110 mg, 110 mg and 120 mg, 120 mg and 125 mg, 125 mg and 130 mg, 130 mg and 135 mg, 135 mg and 140 mg, 140 mg and 145 mg, 145 mg and 150 mg, 150 mg and 155 mg, 155 mg and 160 mg, 160 mg and 165 mg, 165 mg and 170 mg, 170 mg and 175 mg, 175 and 180 mg, 180 mg and 185 mg, 185 mg and 190 mg, 190 mg and 195 mg, 195 mg and 200 mg, 200 mg and 205 mg, 205 mg and 210 mg, 210 mg and 215 mg, 215 mg and 220 mg, 220 mg and 225 mg, 225 mg and 230 mg, 230 mg and 235 mg, 235 mg and 240 mg, 240 mg and 245 mg, 245 mg and 250 mg, 250 mg and 255 mg, 255 mg and 260 mg, 260 mg and 265 mg, 265 mg and 270 mg, 270 mg and 275 mg, 275 and 280 mg, 280 mg and 285 mg, 285 mg and 290 mg, 290 mg and 295 mg, and 295 mg and 300 mg of the muscarinic orthosteric agonist per day. In certain embodiments, the total daily dose of the muscarinic orthosteric agonist is chosen from 25 mg, 50 mg, 60 mg, 75 mg, 90 mg, 100 mg, 120 mg, 150 mg, and 175 mg. In certain embodiments, the total daily dose of the muscarinic orthosteric agonist is at least 25 mg. In certain embodiments, the total daily dose of the muscarinic orthosteric agonist is less than 175 mg.

In certain embodiments, the dosage of xanomeline and/or a salt thereof ranges between 5 mg and 700 mg xanomeline free base per day, such as between 25 mg and 300 mg xanomeline free base per day, such between 25 mg and 30 mg, 30 mg and 35 mg, 35 mg and 40 mg, 40 mg and 45 mg, 45 mg and 50 mg, 50 mg and 55 mg, 55 mg and 60 mg, 60 mg and 65 mg, 65 mg and 70 mg, 70 mg and 75 mg, 75 and 80 mg, 80 mg and 85 mg, 85 mg and 90 mg, 90 mg and 95 mg, 95 mg and 100 mg, 105 mg and 110 mg, 110 mg and 120 mg, 120 mg and 125 mg, 125 mg and 130 mg, 130 mg and 135 mg, 135 mg and 140 mg, 140 mg and 145 mg, 145 mg and 150 mg, 150 mg and 155 mg, 155 mg and 160 mg, 160 mg and 165 mg, 165 mg and 170 mg, 170 mg and 175 mg, 175 and 180 mg, 180 mg and 185 mg, 185 mg and 190 mg, 190 mg and 195 mg, 195 mg and 200 mg, 200 mg and 205 mg, 205 mg and 210 mg, 210 mg and 215 mg, 215 mg and 220 mg, 220 mg and 225 mg, 225 mg and 230 mg, 230 mg and 235 mg, 235 mg and 240 mg, 240 mg and 245 mg, 245 mg and 250 mg, 250 mg and 255 mg, 255 mg and 260 mg, 260 mg and 265 mg, 265 mg and 270 mg, 270 mg and 275 mg, 275 and 280 mg, 280 mg and 285 mg, 285 mg and 290 mg, 290 mg and 295 mg, and 295 mg and 300 mg of xanomeline free base per day. In certain embodiments, the total daily dose of xanomeline is chosen from 25 mg, 50 mg, 60 mg, 75 mg, 90 mg, 100 mg, 120 mg, 150 mg, and 175 mg. In certain embodiments, the total daily dose of xanomeline is at least 25 mg. In certain embodiments, the total daily dose of xanomeline is less than 175 mg.

In certain embodiments, the dosage of the PAM ranges between 0.5 mg and 700 mg PAM per day, such as between 0.5 mg and 300 mg of the PAM, such between 0.5 mg and 1 mg, between 1 mg and 5 mg, between 5 mg and 10 mg, between 10 mg and 15 mg, between 15 mg and 20 mg, between 20 mg and 25 mg, between 25 mg and 30 mg, 30 mg and 35 mg, 35 mg and 40 mg, 40 mg and 45 mg, 45 mg and 50 mg, 50 mg and 55 mg, 55 mg and 60 mg, 60 mg and 65 mg, 65 mg and 70 mg, 70 mg and 75 mg, 75 and 80 mg, 80 mg and 85 mg, 85 mg and 90 mg, 90 mg and 95 mg, 95 mg and 100 mg, 105 mg and 110 mg, 110 mg and 120 mg, 120 mg and 125 mg, 125 mg and 130 mg, 130 mg and 135 mg, 135 mg and 140 mg, 140 mg and 145 mg, 145 mg and 150 mg, 150 mg and 155 mg, 155 mg and 160 mg, 160 mg and 165 mg, 165 mg and 170 mg, 170 mg and 175 mg, 175 and 180 mg, 180 mg and 185 mg, 185 mg and 190 mg, 190 mg and 195 mg, 195 mg and 200 mg, 200 mg and 205 mg, 205 mg and 210 mg, 210 mg and 215 mg, 215 mg and 220 mg, 220 mg and 225 mg, 225 mg and 230 mg, 230 mg and 235 mg, 235 mg and 240 mg, 240 mg and 245 mg, 245 mg and 250 mg, 250 mg and 255 mg, 255 mg and 260 mg, 260 mg and 265 mg, 265 mg and 270 mg, 270 mg and 275 mg, 275 and 280 mg, 280 mg and 285 mg, 285 mg and 290 mg, 290 mg and 295 mg, and 295 mg and 300 mg of the PAM per day. In certain embodiments, the total daily dose of the PAM is chosen from 25 mg, 50 mg, 60 mg, 75 mg, 90 mg, 100 mg, 120 mg, 150 mg, and 175 mg. In certain embodiments, the total daily dose of the PAM is at least 25 mg. In certain embodiments, the total daily dose of the PAM is less than 175 mg.

In certain embodiments, the PAM is GSK 1034702, and the dose is 0.5 mg or 5 mg following a dose-escalation plan. In certain embodiments, the PAM is GSK 1034702, and the dose is chosen from 4 mg or 8 mg.

In certain embodiments, the PAM is MK-7622, and the dose is a single 45 mg capsule once daily, taken orally. In certain embodiments, the PAM is MK-7622 following a dose-escalation plan, such as 15 mg MK-7622 once daily for one week, followed by 30 mg MK-7622 once daily for one week, and then 45 mg MK-7622 once daily for the remainder of the treatment.

In certain embodiments, the PAM is TAK-017, and the dose is 7.5 mg.

In certain embodiments, the PAM is emraclidine, and the dose is chosen from 10 mg, 15 mg, and 30 mg. In certain embodiments, when present, the dosage of the muscarinic agonist ranges between 5 mg and 60 mg muscarinic agonist per day, such as between 5 mg and 10 mg, between 10 mg and 15 mg, between 15 mg and 20 mg, between 20 mg and 25 mg, between 25 mg and 30 mg, 30 mg and 35 mg, 35 mg and 40 mg, 40 mg and 45 mg, 45 mg and 50 mg, 50 mg and 55 mg, and 55 mg and 60 mg per day.

In certain embodiments, the muscarinic agonist is a salt of trospium ranging between 5 mg and 60 mg of the salt of trospium per day, such as between 5 mg and 10 mg, between 10 mg and 15 mg, between 15 mg and 20 mg, between 20 mg and 25 mg, between 25 mg and 30 mg, 30 mg and 35 mg, 35 mg and 40 mg, 40 mg and 45 mg, 45 mg and 50 mg, 50 mg and 55 mg, and 55 mg and 60 mg per day.

In certain embodiments, the muscarinic agonist is a trospium chloride ranging between 5 mg and 60 mg of the salt of trospium chloride, such as between 5 mg and 10 mg, between 10 mg and 15 mg, between 15 mg and 20 mg, between 20 mg and 25 mg, between 25 mg and 30 mg, 30 mg and 35 mg, 35 mg and 40 mg, 40 mg and 45 mg, 45 mg and 50 mg, 50 mg and 55 mg, and 55 mg and 60 mg per day.

In certain embodiments, the administration comprises a dosing schedule of 25 mg xanomeline and/or the salt thereof and 5 mg a salt of trospium twice daily.

In certain embodiments, the administration comprises a dosing schedule of 25 mg xanomeline and/or the salt thereof and 5 mg a salt of trospium thrice daily.

In certain embodiments, the administration comprises a dosing schedule of 25 mg xanomeline and/or the salt thereof and 5 mg a salt of trospium twice daily, and 50 mg xanomeline and/or the salt thereof and 7.5 mg a salt of trospium once daily.

In certain embodiments, the administration comprises a dosing schedule of 25 mg xanomeline and/or the salt thereof and 10 mg a salt of trospium twice daily.

In certain embodiments, the administration comprises a dosing schedule of 50 mg xanomeline and/or the salt thereof and 7.5 mg a salt of trospium twice daily, and 50 mg xanomeline and/or the salt thereof and 5 mg a salt of trospium once daily.

In certain embodiments, the administration comprises a dosing schedule of 50 mg xanomeline and/or the salt thereof and 10 mg a salt of trospium twice daily.

In certain embodiments, the administration comprises a dosing schedule of 50 mg xanomeline and/or the salt thereof and 10 mg a salt of trospium thrice daily.

In certain embodiments, the administration comprises a dosing schedule of 50 mg xanomeline and/or the salt thereof and 10 mg a salt of trospium twice daily, and 75 mg xanomeline and/or the salt thereof and 10 mg a salt of trospium once daily.

In certain embodiments, the administration comprises a dosing schedule of 75 mg xanomeline and/or the salt thereof and 10 mg a salt of trospium twice daily, and 50 mg xanomeline and/or the salt thereof and 10 mg a salt of trospium once daily.

In certain embodiments, the low doses are split TID rather than BID. BID keeps the trospium dose as low as possible with a minimum amount of 10 mg of trospium per capsule. In certain embodiments, a starting dose of 50/5 mg or 50/7.5 mg xanomeline/trospium is split TID so the individual dose 17/2.5 mg xanomeline/trospium.

In certain embodiments, combining the muscarinic orthosteric agonist with a PAM, the desired therapeutic effect is achieved while diminishing or eliminating the side effects of activating muscarinic receptors located outside the brain. Without wishing to be bound by theory, the muscarinic orthosteric agonist directly activates the receptor while the PAM increases the receptors' receptivity to the muscarinic orthosteric agonist.

In certain embodiments, combining xanomeline with a PAM, the desired therapeutic effect is achieved while diminishing or eliminating the side effects of activating muscarinic receptors located outside the brain. Without wishing to be bound by theory, the xanomeline directly activates the receptor while the PAM increases the receptors' receptivity to the xanomeline.

In certain embodiments, the PAM increases the response of the muscarinic receptor by increasing the probability that the orthostatic agonist will bind to a receptor (i.e., affinity), increasing the orthostatic agonist's ability to activate the receptor (i.e., efficacy), or both. Affinity is the ability of a substance to bind to a receptor. Efficacy is the ability of a substance to activate a receptor, given as a percentage of the ability of the substance to activate the receptor compared to the receptor's endogenous agonist.

In certain embodiments, the PAM induces a conformational change in the muscarinic receptor, which increases the binding affinity and/or efficacy of the muscarinic orthosteric agonist, such as the xanomeline and/or salt thereof. In certain embodiments, the PAM stabilizes conformational changes associated with the agonist-bound state. This increases the probability that the muscarinic receptor will be in the active conformation but does not prevent the receptor from switching back to the inactive state. With a higher probability of remaining in the active state, the muscarinic receptor will bind orthosteric agonist for longer.

In certain embodiments, the overall signal is increased by preventing the desensitization of the muscarinic receptor. Desensitization prevents the muscarinic receptor from activating, despite the presence of the orthosteric agonist. This may be caused by repeated or intense exposures to the orthosteric agonist. Eliminating or reducing this phenomenon increases the muscarinic receptor's overall activation.

In certain embodiments, the PAM can directly regulate the muscarinic receptor rather than affecting the binding of the orthosteric agonist. Similar to stabilizing the bound conformation of the muscarinic receptor, the PAM via this mechanism stabilizes a conformation associated with the active or inactive state. This increases the probability that the muscarinic receptor will conform to the stabilized state and modulate the receptor's activity accordingly.

Various methods can demonstrate the efficacy of the combination of the muscarinic orthosteric agonist, such as xanomeline, and a PAM. For example, animal models demonstrate the efficacy of new therapeutics for schizophrenia, including pharmacological models (e.g., ketamine) and genetic models (e.g., DISC1 mouse). Likewise, animal models, including rodents, dogs, and non-human primates, may be used to demonstrate the side effect profile of pharmacological agents.

Alternatively, the disclosed combination can be tried in controlled clinical trials of people. Standard measures based on patient self-report can be used by those skilled in the art to assess various side effects such as GI discomfort. As another example, objective physiological measures (e.g., EKGs) may be used by those skilled in the art. A set of standard measures has also been developed to assess schizophrenia symptoms, including the Brief Psychiatric Rating Scale (BPRS), the Positive and Negative Syndrome Scale (PANSS), and Clinical Global Impression (CGI). Typically, clinical trials are double-blinded, where one group of patients receives an inactive placebo and the other group an active intervention.

In certain embodiments, the patient previously had been administered one or more antipsychotics. In certain embodiments, the patient was an inadequate responder to such administration. In certain embodiments, the patient was treatment resistant.

In certain embodiments, the patient is an adult. In certain embodiments, the patient is elderly, e.g., above the age of 55 years, such as above the age of 65 years. In certain embodiments, the patient has dementia-related psychosis.

In certain embodiments, reducing the daily dose of the PAM improves the overall tolerability of the muscarinic orthostatic agonist. In certain embodiments, the PAM allowed the daily dose of the muscarinic orthostatic agonist to be reduced, thereby allowing for better tolerability. In certain embodiments, the dose of the muscarinic orthostatic agonist and/or a salt thereof or the dose of PAM or both doses is lowered by at least about 5% compared to monotherapy, such as by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, or at least about 75% compared to monotherapy.

In certain embodiments, reducing the daily dose of the PAM improves the overall tolerability of the xanomeline and/or a salt thereof. In certain embodiments, the PAM allowed the daily dose of the xanomeline and/or a salt thereof to be reduced, thereby allowing for better tolerability. In certain embodiments, the dose of the xanomeline and/or a salt thereof or the dose of PAM or both doses is lowered by at least about 5% compared to monotherapy, such as by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, or at least about 75% compared to monotherapy.

In certain embodiments, before the administration, the patient meets clinical criteria for possible or probable Alzheimer's disease.

In certain embodiments, before the administration, the patient has a history of psychotic symptoms meeting International Psychogeriatric Association criteria.

In some embodiments, the treatment occurs outside of a clinical trial setting.

In certain embodiments, the disorder is chosen from dementia-related psychosis, schizophrenia, Alzheimer's disease, Parkinson's disease, depression, movement disorders, pain, drug addiction, tauopathy, and synucleinopathy. In certain embodiments, the disorder is dementia-related psychosis, and the dementia-related psychosis is due to Alzheimer's disease, Lewy body dementia, vascular dementia, dementia related to Parkinson's disease, frontotemporal dementia, or another form of dementia.

In certain embodiments, the patient is treated for at least seven days.

In certain embodiments, the administration is twice daily.

### Pharmaceutical Compositions

Provided herein is a pharmaceutical composition comprising a muscarinic orthostatic agonist, such as xanomeline, and/or salt thereof and a PAM that operates on the same muscarinic receptor subtype. In certain embodiments, the pharmaceutical composition further comprises a muscarinic antagonist, such as trospium chloride.

In certain embodiments, the muscarinic orthostatic agonist and/or a salt thereof and the PAM are in the same pharmaceutical composition.

In certain embodiments, the xanomeline and/or a salt thereof and the PAM are in the same pharmaceutical composition.

In certain embodiments, the muscarinic orthostatic agonist and/or a salt thereof and the muscarinic antagonist are in the same pharmaceutical composition.

In certain embodiments, the xanomeline and/or a salt thereof and the trospium chloride are in the same pharmaceutical composition.

In certain embodiments, the muscarinic antagonist and the PAM are in the same pharmaceutical composition.

In certain embodiments, the muscarinic orthosteric agonist, the muscarinic antagonist and the PAM are in the same pharmaceutical composition.

In certain embodiments, the muscarinic orthostatic agonist and/or a salt thereof is in a first pharmaceutical composition, and the PAM is in a second pharmaceutical composition. In certain embodiments, the xanomeline and/or a salt thereof is in a first pharmaceutical composition, and the PAM is in a second pharmaceutical composition. In certain embodiments, the first composition or the second composition, or both, are co-administered in a reduced amount compared to the first composition or the second composition used alone.

The medicament may also include one or more pharmaceutically acceptable salts. The medicament may include one or more pharmaceutically acceptable carriers. The medicament may be administered orally. The medicament may be delivered orally using tablets, troches, liquids, emulsions, suspensions, drops, capsules, caplets or gel caps, and other methods of oral administration known to one skilled in the art.

The medicament may be in a dosage form that immediately releases the drug. In an alternative embodiment, the medicament may have a controlled release dosage form.

The medicament may be in dosage forms that use other controlled-release formulations known to one in the art.

In another embodiment, the medicament is combined with one or more therapies, including psychotherapy and drugs. Therapeutic agents include, but are not limited to, antipsychotics, anxiolytics, antidepressants, sedatives, tranquilizers, analgesics, and other pharmacological interventions known to one skilled in the art. A therapeutic agent may fall under the category of more than one drug. For instance, benzodiazepines can be considered anxiolytics, sedatives, and tranquilizers.

In certain embodiments, the pharmaceutical composition further comprises an anticholinergic agent. In certain embodiments, the anticholinergic agent is a muscarinic antagonist. In certain embodiments, the muscarinic antagonist is chosen from trospium, tolterodine, darifenacin, solifenacin, fesoterodine, scopolamine, N-methylscopolamine, and a salt thereof.

In certain embodiments, the muscarinic antagonist is a salt of trospium. In certain embodiments, the salt of trospium is chosen from trospium chloride, trospium bromide, trospium iodide, and trospium saccharinate. In certain embodiments, the muscarinic antagonist is trospium chloride.

In certain embodiments, the muscarinic antagonist and the orthosteric muscarinic agonist are administered in a first composition and the PAM is administered in a second composition. In one embodiment, the excipients include one or more fillers, binders, and surfactants. Other optional ingredients include, but are not limited to, glidants, lubricants, disintegrants, swelling agents, and antioxidants.

The amount of the muscarinic orthostatic agonist can be at least 10 wt. % or at least 15 wt. %, or at least 20 wt. %, or at least 25 wt. %, or at least 30 wt. %. For example, the amount of the muscarinic orthostatic agonist can be at least 50 wt. %, or at least 55 wt. %, or at least 60 wt. %, or at least 65 wt. %, or at least 70 wt. %, or at least 75 wt. %, or at least 80 wt. %, or at least 85 wt. % of the pharmaceutical composition, in a range of about 60 wt. % to about 90 wt. % or about 65 wt. % to about 85 wt. %. It is understood that all ranges, including these values as endpoints, are contemplated, for example, at least between about 15 wt. % and about 90 wt. %, between about 20 wt. % and about 85 wt. %, between about 30 wt. % and about 85 wt. %, or between about 50 wt. % and about 90 wt. %. In certain embodiments, the pharmaceutical composition comprises between 30 wt. % and 80 wt. % muscarinic orthostatic agonist, such as 66 wt. % muscarinic orthostatic agonist.

The amount of xanomeline free base can be at least 10 wt. % or at least 15 wt. %, or at least 20 wt. %, or at least 25 wt. %, or at least 30 wt. %. For example, the amount of xanomeline tartrate can be at least 50 wt. %, or at least 55 wt. %, or at least 60 wt. %, or at least 65 wt. %, or at least 70 wt. %, or at least 75 wt. %, or at least 80 wt. %, or at least 85 wt. % of the pharmaceutical composition, in a range of about 60 wt. % to about 90 wt. % or about 65 wt. % to about 85 wt. %. It is understood that all ranges, including these values as endpoints, are contemplated, for example, at least between about 15 wt. % and about 90 wt. %, between about 20 wt. % and about 85 wt. %, between about 30 wt. % and about 85 wt. %, or between about 50 wt. % and about 90 wt. %. In certain embodiments, the pharmaceutical composition comprises between 30 wt. % and 80 wt. % xanomeline tartrate, such as 66 wt. % xanomeline tartrate.

In a further embodiment, the pharmaceutical composition comprises a polymer, for example, to modify the release profile of the active. In a further embodiment, the polymer comprises a water-soluble polymer. In a further embodiment, the water-soluble polymer is selected from Eudragit™ RL, polyvinyl alcohol, polyvinylpyrrolidone, methylcellulose, hydroxypropyl cellulose, hydroxypropylmethylcellulose, polyethylene glycol, and mixtures thereof. In a further embodiment, the polymer comprises a water-insoluble polymer. In a further embodiment, the water-insoluble polymer is selected from Eudragit™ RS, ethylcellulose, cellulose acetate, cellulose propionate, cellulose acetate propionate, cellulose acetate butyrate, cellulose acetate phthalate, cellulose triacetate, poly(methyl methacrylate), poly(ethyl methacrylate), poly(butyl methacrylate), poly(isobutyl methacrylate), poly(hexyl methacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate), poly(ethylene), poly(ethylene) low density, poly(ethylene) high density, poly(propylene), poly(ethylene terephthalate), poly(vinyl isobutyl ether), poly(vinyl acetate), poly(vinyl chloride), polyurethane, and mixtures thereof.

Fillers include, but are not limited to, lactose, saccharose, glucose, starch, microcrystalline cellulose, microfine cellulose, mannitol, sorbitol, calcium hydrogen phosphate, aluminum silicate, amorphous silica, sodium chloride, starch, and dibasic calcium phosphate dihydrate. In one embodiment, the filler is not water-soluble, although it may absorb water. In one embodiment, the filler is a spheronization aid. Spheronization aids can include one or more of crospovidone, carrageenan, chitosan, pectinic acid, glycerides, β-cyclodextrin (β-CD), cellulose derivatives, microcrystalline cellulose, powdered cellulose, polyplasdone crospovidone, and polyethylene oxide. In one embodiment, the filler includes microcrystalline cellulose.

The amount of filler is not particularly limited. In embodiments, the amount of filler (e.g., microcrystalline cellulose) can range from about 10 wt. % to about 70 wt. %, or about 16 wt. % to about 23 wt. %, or at least 19 wt. % or at least 19.5 wt. %, for example, about 20 wt. %.

Binders include, but are not limited to, cellulose ethers, methylcellulose, ethylcellulose, hydroxyethylcellulose, propyl cellulose, hydroxypropyl cellulose, lower-substituted hydroxypropyl cellulose, hydroxypropylmethylcellulose (hypromellose, e.g., hypromellose 2910, Methocel™ E), carboxymethyl cellulose, starch, pregelatinized starch, acacia, tragacanth, gelatin, polyvinyl pyrrolidone (povidone), cross-linked polyvinyl pyrrolidone, sodium alginate, microcrystalline cellulose, and lower-alkyl-substituted hydroxypropyl cellulose. In one embodiment, the binders are selected from wet binders. In one embodiment, the binder is selected from cellulose ethers, e.g., hypromellose.

The amount of binder is not particularly limited. In embodiments, the binder (e.g., hypromellose) can be between about 1 wt. % and about 10 wt. %, between about 2 wt. % and about 8 wt. %, or between about 4 wt. % and about 6 wt. %, for example, about 5 wt. %.

Surfactants include, but are not limited to, anionic surfactants, including sodium lauryl sulfate, sodium deoxycholate, dioctyl sodium sulfosuccinate, and sodium stearyl fumarate, nonionic surfactants, including polyoxyethylene ethers and polysorbate 80, and cationic surfactants, including quaternary ammonium compounds. In one embodiment, the surfactant is selected from anionic surfactants, e.g., sodium lauryl sulfate.

The amount of surfactant, e.g., as a processing aid, is not particularly limited. In embodiments, surfactant (e.g., microcrystalline cellulose) can range between about 0.1 wt. % and about 1 wt. %, between about 0.2 wt. % and about 0.8 wt. %, or between about 0.4 wt. % and about 0.6 wt. %, for example, about 0.5 wt. %.

Disintegrants include, but are not limited to, starch, sodium cross-linked carboxymethyl cellulose, carmellose sodium, carmellose calcium, cross-linked polyvinyl pyrrolidone, sodium starch glycolate, low-substituted hydroxypropyl cellulose, and hydroxypropyl starch.

Glidants include but are not limited to polyethylene glycols of various molecular weights, magnesium stearate, calcium stearate, calcium silicate, fumed silicon dioxide, magnesium carbonate, magnesium lauryl sulfate, aluminum stearate, stearic acid, palmitic acid, cetanol, stearol, and talc.

Lubricants include, but are not limited to, stearic acid, magnesium stearate, calcium stearate, aluminum stearate, and siliconized talc. In certain embodiments, the pharmaceutical composition comprises between 0 wt. % and 2 wt. % talc, such as 0.5 wt. % talc.

In certain embodiments, the formulation further comprises one or more antioxidants. Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like. In certain embodiments, the formulation comprises less than 1 wt. % antioxidant, such as 0.9 wt. %, 0.8 wt. %, 0.7 wt. %, 0.6 wt. %, 0.5 wt. %, 0.4 wt. %, 0.3 wt. %, 0.2 wt. %, 0.1 wt. %, 0.09 wt. %, 0.08 wt. %, 0.07 wt. %, 0.06 wt. %, 0.05 wt. %, 0.04 wt. %, 0.03 wt. %, 0.02 wt. %, or 0.01 wt. %.

In certain embodiments, the oral pharmaceutical composition further comprises ascorbic acid. In certain embodiments, the oral pharmaceutical composition comprises between 0.2 wt. % and 1 wt. % ascorbic acid. In certain embodiments, the oral pharmaceutical composition comprises about 0.5 wt. % ascorbic acid. In certain embodiments, the oral pharmaceutical composition further comprises butylated hydroxytoluene. In certain embodiments, the oral pharmaceutical composition comprises between 0.01 wt. % and 0.1 wt. % butylated hydroxytoluene. In certain embodiments, the oral pharmaceutical composition comprises about 0.05 wt. % butylated hydroxytoluene. In certain embodiments, the formulation comprises about 0.05 wt. % BHT or 0.5 wt. % ascorbic acid.

Depending on dosing requirements, capsules can be prepared with different amounts of muscarinic orthosteric agonist, such as xanomeline and/or a salt thereof, and the PAM.

In other embodiments, the pharmaceutical composition may be coated with functional or non-functional coatings, such as aesthetic, handling, or stability. In certain embodiments, the pharmaceutical composition might be coated with a pH-sensitive coating so that they do not dissolve in the low pH of the stomach. A non-functional coating might maintain chemical separation between the pharmaceutical compositions or cosmetic reasons. In certain embodiments, the distribution of coating thicknesses can be stated in the weight gain of coating material based on the total weight of the pharmaceutical composition. For example, the difference in coating thickness from composition to composition can be in a range of +/−1-7% based on the total weight of the pharmaceutical composition.

The enteric (gastro-resistant) coating material, e.g., polymer, can be one that will dissolve in intestinal juices at a pH level higher than that of the stomach, e.g., a pH of greater than 4.5, such as within the small intestine, and therefore permit the release of the active substance in the regions of the small intestine and substantially not in the upper portion of the GI tract. In one embodiment, the enteric material begins to dissolve in an aqueous solution at pH between about 4.5 and about 5.5. In another embodiment, the enteric material rapidly dissolves in an aqueous solution at a pH of about 5. In another embodiment, the enteric material rapidly dissolves in an aqueous solution at a pH of about 5.5.

For example, pH-sensitive materials do not dissolve significantly until the dosage form empties from the stomach. The small intestine's pH gradually increases from about 4.5 to about 6.5 in the duodenal bulb to about 7.2 in the small intestine's distal portions (ileum). To provide predictable dissolution corresponding to the small intestine transit time of about 3 hours (e.g., 2-3 hours) and permit reproducible release therein, the coating should begin to dissolve within the pH range of the duodenum and continue to dissolve at the pH range within the small intestine. Therefore, the amount (thickness) of enteric coating should be substantially dissolved during about three hours within the small intestine (e.g., the proximal and mid-small intestine).

Suitable enteric (gastro-resistant) materials include, but are not limited to, cross-linked polyvinyl pyrrolidone; non-crosslinked polyvinylpyrrolidone; hydroxypropylmethyl cellulose phthalate, hydroxypropylmethyl cellulose acetate succinate, cellulose acetate succinate; cellulose acetate phthalate, hydroxypropylmethyl cellulose acetate succinate, cellulose acetate trimellitate; starch acetate phthalate; polyvinyl acetate phthalate; carboxymethyl cellulose; methyl cellulose phthalate; methyl cellulose succinate; methyl cellulose phthalate succinate; methyl cellulose phthalic acid half ester; ethyl cellulose succinate; carboxymethylamide; potassium methacrylate divinylbenzene copolymer; polyvinyl alcohols; polyoxyethylene glycols; polyethylene glycol; sodium alginate; galactomannan; carboxypolymethylene; sodium carboxymethyl starch; copolymers of acrylic acid and/or methacrylic acid with a monomer selected from the following: methyl methacrylate, ethyl methacrylate, ethyl acrylate, butyl methacrylate, hexyl methacrylate, decyl methacrylate, lauryl methacrylate, phenyl methacrylate, methyl acrylate, isopropyl acrylate, isobutyl acrylate, or octadecyl acrylate, e.g. Eudragit™-L and -S series, including L 100-55, L 30 D-55, L 100, S 100, L 12.5, and S 12.5, available from Evonik Industries; polyvinyl acetate; fats; oils; waxes; fatty alcohols; shellac; zein; gluten; ethylacrylate-maleic acid anhydride copolymer; maleic acid anhydride-vinyl methyl ether copolymer; styrol-maleic acid copolymer; 2-ethyl-hexyl-acrylate maleic acid anhydride; crotonic acid-vinyl acetate copolymer; glutaminic acid/glutamic acid ester copolymer; carboxymethylethylcellulose glycerol monooctanoate; polyarginine; poly(ethylene); poly(propylene); poly(ethylene oxide); poly(ethylene terephthalate); poly(vinyl isobutyl ether); poly(vinyl chloride); and polyurethane.

A combination of enteric materials may also be used. In one embodiment, the enteric material rapidly dissolves at pH 5.5 and higher to provide fast dissolution in the upper bowel. For example, the enteric material can be selected from a copolymer of methacrylic acid and methyl methacrylate and a copolymer of methacrylic acid and ethyl acrylate. For example, an enteric polymer is poly(methacrylic acid co-ethyl acrylate)1:1 (Eudragit™ L 30 D-55 and Eudragit™ L 100-55).

Other suitable examples of enteric coating coatings include beeswax and glyceryl monostearate; beeswax, shellac, and cellulose; and cetyl alcohol, mastic and shellac, and shellac and stearic acid; polyvinyl acetate and ethyl cellulose; and a neutral copolymer of polymethacrylic acid esters (Eudragit™ L 30D); copolymers of methacrylic acid and methacrylic acid methylester, or a neutral copolymer of polymethacrylic acid esters containing metallic stearates. Such coatings comprise fats and fatty acids, shellac and shellac derivatives, and cellulose acid phthalates, e.g., those with free carboxyl content.

One or more plasticizers can be added to enteric polymers to increase their pliability and reduce brittleness, as known in the art. Suitable plasticizers include, for example, butyl citrates, triethyl citrate, diethyl phthalate, dibutyl sebacate, polyethylene glycols (PEGs, such as PEG 6000), acetyl triethyl citrate, and triacetin. In one embodiment, the plasticizer is triethyl citrate. While some enteric materials are flexible and do not require plasticizers, more brittle polymers (e.g., Eudragit™ L/S types, Eudragit™ RL/RS, and Eudragit™ FS 30 D) benefit from plasticizers, for example, ranging from between 5 wt. % and 30 wt. % based on the dry polymer mass, between about 8 wt. % and about 12 wt. % triethyl citrate with poly(methacrylic acid co-ethyl acrylate) 1:1.

In certain embodiments, the enteric coatings comprise one or more anti-tacking agents (antiadherents) to reduce the film's tackiness and prevent agglomeration, as it is known in the art. Suitable anti-tacking agents include, but are not limited to, talc, glyceryl monostearate, fumed silica (e.g., Aerosil™ 200), precipitated silica (e.g., Sipernat™ PQ), and magnesium stearate. Anti-tacking agents can be used in any suitable quantity, for example ranging between about 10 wt. % and 100 wt. % based on dry polymer mass, between about 10 wt. % and about 50 wt. %, between about 10 wt. % and about 30 wt. %, or between about 15 wt. % and about 30 wt. %. For example, in one embodiment, it ranges between 15 wt. % and about 30 wt. % based on dry polymer mass.

One or more surfactants can also be added to an enteric coating mixture to increase substrate wettability and/or stabilize suspensions, as it is known in the art. Surfactants include Polysorbate 80, sorbitan monooleate, sodium dodecyl sulfate, and other surfactants described herein.

Any suitable process can form the enteric coating. Coating processes include pan coating, fluid bed coating, and dry coating (e.g., heat dry coating and electrostatic dry coating). Pan coating and fluid bed coating using solvent are well-established processes. In liquid coating, the enteric material and optional excipients (e.g., pigments, plasticizers, anti-tacking agents) are mixed in an organic solvent or water to form a solution or dispersion. The coating solution or dispersion is sprayed into solid dosage forms in a pan coater or a fluid bed dryer and dried by hot air. For example, in a Wurster fluid bed coating process, the coating fluid is sprayed from the fluid bed apparatus's bottom. Alternatively, the coating fluid is applied by top spraying. In certain embodiments, a tangential spray is applied.

The enteric material applied is sufficient to achieve the desired acid resistance and release characteristics. For example, in one embodiment, the amount of enteric coating meets USP <711> requirements (USP 36-NF 31) for delayed-release dosage forms, thereby not releasing 10.0 wt. % of the drug after 2 hours in 0.1 N HCl. In certain embodiments, the formulation releases at least 80% of the active in 20 minutes in pH 6.8 buffer solution, e.g., using a dissolution method of USP 36-NF 31 section <711>.

In one embodiment, the enteric coating is present in an amount in a range between about 10% and 40%, or between 25% and about 35% as measured by the weight gain compared to the uncoated pharmaceutical composition, or ranging between about 25% and about 31% weight gain, between about 27% and about 31% weight gain, or between about 28.5% and about 31% weight gain, based on the weight of the uncoated pharmaceutical composition.

The formulation can include a capsule shell. Soft and hard capsule shells are known. The capsule shell is a hard-capsule shell in one embodiment, e.g., a gelatin capsule shell or a vegetable-based hard capsule shell. In certain embodiments, the capsule shell comprises one or more enteric coatings described herein. During accelerated storage, gelatin capsules may collapse. Thus, in certain embodiments, the formulation can include a hydroxypropyl methylcellulose capsule shell.

In certain embodiments, the excipient is chosen from microcrystalline cellulose, lactose, and talc.

In certain embodiments, the pharmaceutical composition is formulated for oral administration. In certain embodiments, the pharmaceutical composition is in the form of a capsule.

In certain embodiments, the pharmaceutical composition is stored with a desiccant, for example, pharmaceutical grades of silica gel, crystalline sodium, potassium or calcium aluminosilicate, colloidal silica, anhydrous calcium sulfate, and the like.

In certain embodiments, the pharmaceutical composition is stored with an oxygen absorber.

In certain embodiments, the pharmaceutical composition is stored under a dry inert gas such as nitrogen, helium, argon, neon, xenon, krypton, or a mixture thereof.

In certain embodiments, the pharmaceutical composition is stored under reduced pressure compared to the external ambient air.

In certain embodiments, the pharmaceutical composition is stored at a reduced temperature, e.g., at refrigerated temperatures (e.g., 2° C. to 8° C.).

In certain embodiments, the pharmaceutical composition is stored by a manufacturer, a distributor, a pharmacy, or a hospital at a temperature of between about 2° C. and about 8° C. before dispensing the oral pharmaceutical composition to the subject. In certain embodiments, after the oral pharmaceutical composition is dispensed to the subject, the pharmaceutical composition is stored at a temperature of between about 20° C. and about 25° C.

In certain embodiments, the total impurities in the pharmaceutical compositions provided herein are no greater than about 5% by weight, no greater than about 4% by weight, no greater than about 3% by weight, no greater than about 2.5% by weight, no greater than about 2% by weight, no greater than about 1.5% by weight, no greater than about 1% by weight, no greater than about 0.5% by weight, or no greater than about 0.1% by weight.

### Definitions

The articles “a” and “an” refer to one or more than one (i.e., to at least one) of the grammatical object of the article. By way of example, “an element” means one element or more than one element.

The terms “comprise” and “comprising” are inclusive, open sense, meaning that additional elements may be included.

The term “consisting of” limits the elements to those specified except for impurities ordinarily associated in addition to that.

The term “consisting essentially of” limits those specified elements and those that do not materially affect the basic and novel characteristics of the material or steps.

All ranges set forth herein include all possible subsets of ranges and any combinations of such subset ranges. By default, ranges include the stated endpoints, unless stated otherwise, where a range of values is provided, each intervening value between the upper and lower limit of that range and any other stated or intervening value in that stated range is encompassed within the disclosure. The upper and lower limits of these smaller ranges may independently be included in the smaller ranges and encompassed within the disclosure, subject to any specifically excluded limit in the stated range. Where the stated range includes one or both limits, ranges excluding either or both of those included limits are also contemplated to be part of the disclosure.

The term “wt. %” is the weight percent based on the total weight, as described in context. Unless stated otherwise, the wt. % is intended to describe the weight percent based on the dry weight.

The term “controlled release” is defined as a prolonged-release pattern of one or more drugs, such that the drugs are released over a period. A controlled release formulation has release kinetics that results in measurable serum levels of the drug over a period longer than what would be possible following intravenous injection or following administration of an immediate release oral dosage form. Controlled release, slow-release, sustained-release, extended-release, prolonged-release, and delayed-release have the same definitions.

The term “including” means “including but not limited to.” “Including” and “including but not limited to” are interchangeable.

The term “mammal” is known in the art. Exemplary mammals include humans, primates, bovines, porcines, canines, felines, and rodents (e.g., mice and rats).

A “patient,” “subject,” or “host” to be treated by the subject method means either a human or non-human mammal.

The term “pharmaceutically acceptable carrier” is art-recognized. It refers to a pharmaceutically acceptable material, composition, or vehicles, such as a liquid or solid filler, diluent, excipient, solvent, or encapsulating material, involved in carrying or transporting any subject composition or component thereof from one organ, or portion of the body, to another organ, or portion of the body. Each carrier must be “acceptable” in the sense of being compatible with the subject composition and its components and not injurious to the patient.

Some examples of materials that may serve as pharmaceutically acceptable carriers include sugars, such as lactose, glucose, and sucrose; starches, such as corn starch and potato starch; cellulose and its derivatives, such as sodium carboxymethyl cellulose, ethylcellulose, and cellulose acetate; powdered tragacanth; malt; gelatin; talc; excipients, such as cocoa butter and suppository waxes; oils, such as peanut oil, cottonseed oil, safflower oil, sesame oil, olive oil, corn oil, and soybean oil; glycols, such as propylene glycol; polyols, such as glycerin, sorbitol, mannitol, and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations.

The term “pharmaceutically acceptable salt” or “salt” is art-recognized. It refers to a salt prepared from relatively non-toxic acids or bases, including inorganic acids and bases and organic acids and bases, including, for example, those contained in compositions of the present disclosure. Suitable non-toxic acids include inorganic and organic acids such as acetic, benzenesulfonic, benzoic, camphorsulfonic, citric, ethenesulfonic, fumaric, gluconic, glutamic, hydrobromic, hydrochloric, isethionic, lactic, maleic, malic, mandelic, methanesulfonic, mucic, nitric, pamoic, pantothenic, phosphoric, saccharinate, succinic, sulfuric, tartaric acid, p-toluenesulfonic, hydrochloric, hydrobromic, phosphoric, and sulfuric acids and the like.

“Treating” is art-recognized and refers to curing and ameliorating at least one symptom of any condition or disorder.

In jurisdictions that forbid the patenting of methods practiced on the human body, the meaning of “administering” a composition to a human subject shall be restricted to prescribing a controlled substance that a human subject will self-administer by any technique (e.g., orally, inhalation, topical application, injection, insertion, etc.). The broadest reasonable interpretation consistent with laws or regulations defining patentable subject matter is intended. In jurisdictions that do not forbid the patenting of methods practiced on the human body, the “administering” of compositions includes both methods practiced on the human body and the foregoing activities.

The term “therapeutic agent” is art-recognized and refers to any chemical moiety that is a biologically, physiologically, or pharmacologically active substance acting locally or systemically in a subject. Examples of therapeutic agents, also referred to as “drugs,” are described in well-known literature references such as the Merck Index (14th edition), the Physicians' Desk Reference (64th edition), and The Pharmacological Basis of Therapeutics (12th edition). These therapeutic agents include, without limitation, medicaments; vitamins; mineral supplements; substances used for the treatment, prevention, diagnosis, cure, or mitigation of a disease or illness; substances that affect the structure or function of the body, or pro-drugs, which become biologically active or more active after they have been placed in a physiological environment.

The term “inhibitor” means a molecule described as an antagonist, partial antagonist, competitive antagonist, non-competitive antagonist, uncompetitive antagonist, silent antagonist, inverse agonist, reversible antagonist, physiological antagonist, irreversible antagonist, inhibitor, reversible inhibitor, irreversible inhibitor, negative allosteric modulator, allosteric antagonist, or a molecule that decreases the activity or signaling of receptors directly or indirectly.

As used herein, a “dose” means the measured quantity of an active agent to be taken at one time by a patient. In certain embodiments, wherein the active agent is not xanomeline free base, the quantity is the molar equivalent to the corresponding amount of xanomeline free base. For example, a drug is often packaged in a pharmaceutically acceptable salt form, such as xanomeline tartrate. The dose refers to the mass of the molar equivalent of the corresponding free base xanomeline. For example, 76 mg of xanomeline tartrate is the molar equivalent of 50 mg of xanomeline free base. Likewise, in certain embodiments, wherein the active agent is a PAM salt, the dose is the molar equivalent to the corresponding amount of PAM free base or PAM free acid.

“Cooperation” or “cooperativity” refers to the interaction of one molecule at a receptor protein to affect the binding of another molecule at that receptor protein. For example, some proteins comprise more than one unit and have multiple binding sites. These proteins exhibit “cooperative binding.” When one molecule binds to one binding site at a subunit interface, the protein changes its shape (conformation) to affect binding, either increasing or decreasing the binding affinity at other interfaces. In some instances, cooperative binding leads to an augmented therapeutic effect.

“Efficacy” refers to the ability of a drug to elicit a physiologic response when it interacts with a receptor. “Potency” refers to the amount of drug to produce a certain response. When efficacy and/or potency are increased, less drug elicits an equivalent therapeutic effect.

“Psychotherapy” refers to non-pharmacological therapies. Those skilled in the art use various techniques involving verbal and other interactions with a patient to affect a positive therapeutic outcome. Such techniques include, but are not limited to, behavior therapy, cognitive therapy, psychodynamic therapy, psychoanalytic therapy, group therapy, family counseling, art therapy, music therapy, vocational therapy, humanistic therapy, existential therapy, transpersonal therapy, client-centered therapy (also called person-centered therapy), Gestalt therapy, biofeedback therapy, rational emotive behavioral therapy, reality therapy, response-based therapy, Sandplay therapy, status dynamics therapy, hypnosis, and validation therapy. Psychotherapy may involve combining two or more techniques. A therapist can select and adjust the techniques based on the individual patient's needs and responses.

“Muscarinic disorder” refers to any disease or condition ameliorated by activating the muscarinic system. Such diseases include ones in which direct activation of muscarinic receptors themselves or inhibition of cholinesterase enzymes has produced a therapeutic effect.

“Diseases related to schizophrenia” and “disorders related to schizophrenia” include, but are not limited to, schizo-affective disorder, psychosis, including acute psychosis, delusional disorders, psychosis associated with Alzheimer's disease, psychosis associated with Parkinson's disease, psychotic depression, bipolar disorder, bipolar with psychosis, Huntington's disease, Lewy Body dementia, or any other disease with psychotic features.

“Psychosis” refers to an abnormal condition of the mind that results in difficulties determining what is real and what is not. Symptoms of psychosis include, but are not limited to, false beliefs (delusions), seeing or hearing things that others do not see or hear (hallucinations), incoherent speech, behavior that is inappropriate for the situation, sleep problems, social withdrawal, lack of motivation, and difficulties carrying out daily activities.

“Acute psychosis” refers to the quick or strong onset of psychotic symptoms in a patient, for example, as defined in “Acute and Transient Psychotic Disorder” (International Classification of Diseases-10) and “Brief Psychosis” (DSM-IV). A sharp striking delusion with quick changes in the structure occurs in the individual who has acute psychosis after a short preliminary period of anxiety, insomnia, and confusion. Acute psychosis can include acute psychotic exacerbation when a patient may respond to hallucinations or delusions. Acute psychosis lasts for a short time, typically from one to two weeks.

“Dementia-related psychosis” or “DRP” refers to a common symptom in people with dementia, whether due to Alzheimer's disease, Lewy body dementia, vascular dementia, dementia related to Parkinson's disease, frontotemporal dementia, other forms of dementia, or related disorders. DRP refers to behaviors that can include hallucinations, delusional thinking, agitation, or aggressive behavior. These patients may have visual, auditory, and olfactory hallucinations-seeing, hearing, and smelling things that are not there—or paranoid delusions, such as suspecting a caregiver of wanting to harm them. While psychosis can be more common as dementia advances, visual hallucinations can be an early symptom of Lewy body dementia and Parkinson's-related dementia.

An “adverse event” is any untoward medical occurrence associated with treatment with a pharmaceutical composition described herein. A “mild adverse event” is easily tolerated by the subject, causes minimal discomfort, and does not interfere with everyday activities. A “moderate adverse event” is sufficiently discomforting to interfere with everyday activities; intervention may be needed. A “severe adverse event” prevents everyday activities; treatment or other intervention is usually needed. A “serious adverse event” results in death; is life-threatening (immediate risk of death from the event as it occurred); requires or prolongs inpatient hospitalization; results in persistent or significant disability/incapacity; or results in a congenital anomaly/disability, cancer, or drug overdose. An adverse event is incapacitating or disabling if it results in a substantial or permanent disruption of the subject's ability to carry out normal life functions.

As used herein, a patient is said to “tolerate” a dose of a compound if administering that dose to that patient does not result in an unacceptable adverse event or an unacceptable combination of adverse events. One of skill in the art will appreciate that tolerance is a subjective measure and that what may be tolerable to one patient may not be tolerable to a different patient. For example, one patient may not be able to tolerate a headache. In contrast, a second patient may find headaches tolerable but cannot tolerate vomiting. For a third patient, either headache alone or vomiting alone is tolerable. Still, the patient cannot tolerate the combination of headache and vomiting, even if the severity of each is less than when experienced alone.

The term “maximum tolerated dose” means the highest dose of a drug or therapeutic that a patient can take without experiencing intolerable side effects. The maximum tolerated dose is typically determined empirically in clinical trials.

“Effective amount” and “therapeutically effective amount” of an agent, compound, drug, composition, or combination is an amount that is nontoxic and effective for producing some desired therapeutic effect upon administration to a subject or patient (e.g., a human subject or patient). The precise therapeutically effective amount for a subject may depend upon, e.g., the subject's size and health, the nature and extent of the condition, the therapeutics or combination of therapeutics selected for administration, and other variables known to those of skill in the art. The effective amount for a given situation is determined by routine experimentation and is within the clinician's judgment.

“Informing” means referring to or providing published material, for example, providing an active agent with published material to a user; or presenting information orally, for example, by the presentation at a seminar, conference, or other educational presentation, by the conversation between a pharmaceutical sales representative and a medical care worker, or by the conversation between a medical care worker and a patient; or demonstrating the intended information to a user for comprehension.

“Medication Guide” means an FDA-approved patient labeling for a pharmaceutical product conforming to the specifications set forth in 21 CFR § 208 and other applicable regulations, which contains information for patients on how to safely use a pharmaceutical product. A medication guide is scientifically accurate and is based on, and does not conflict with, the approved professional labeling for the pharmaceutical product under 21 CFR § 201.57, but the language need not be identical to the sections of approved labeling to which it corresponds. A medication guide is typically available for a pharmaceutical product with special risk management information.

A “patient package insert” means information for patients on how to safely use a pharmaceutical product that is part of the FDA-approved labeling. It is an extension of the professional labeling for a pharmaceutical product that may be distributed to a patient when it is dispensed, providing consumer-oriented information about the product in lay language. For example, it may describe benefits, risks, recognizing risks, dosage, or administration.

A “product” or “pharmaceutical product” means a dosage form of an active agent plus published material and optionally packaging.

“Product insert” means the professional labeling (prescribing information) for a pharmaceutical product, a patient package insert for the pharmaceutical product, or a medication guide for the pharmaceutical product.

“Professional labeling” or “prescribing information” means the official description of a pharmaceutical product approved by a regulatory agency (e.g., FDA or EMEA) regulating the marketing of the pharmaceutical product, which includes a summary of the essential scientific information needed for the safe and effective use of the drug, such for example indication and usage; dosage and administration; who should take it; adverse events (side effects); instructions for use in special populations (pregnant women, children, geriatric, etc.); safety information for the patient, and the like.

“Published material” means a medium providing information, including printed, audio, visual, or electronic medium, for example, a flyer, an advertisement, a product insert, printed labeling, an internet website, an internet web page, an internet pop-up window, a radio or television broadcast, a compact disk, a DVD, an audio recording, or other recording or electronic medium.

“Risk” means the probability or chance of an adverse reaction, injury, or other undesirable outcome arising from medical treatment. An “acceptable risk” means measuring the risk of harm, injury, or disease arising from a medical treatment that an individual or group will tolerate. Whether a risk is “acceptable” will depend upon the advantages that the individual or group perceives to be obtainable in return for taking the risk, whether they accept whatever scientific and other advice is offered about the magnitude of the risk, and numerous other factors, both political and social. An “acceptable risk” of an adverse reaction means that an individual or a group in society is willing to take or be subjected to the risk that the adverse reaction might occur since the adverse reaction is one whose probability of occurrence is small or whose consequences are so slight, or the benefits (perceived or real) of the active agent are so great. An “unacceptable risk” of an adverse reaction means that an individual or a group in society is unwilling to take or be subjected to the risk that the adverse reaction might occur upon weighing the probability of occurrence of the adverse reaction, the consequences of the adverse reaction, and the benefits (perceived or real) of the active agent. “At-risk” means a state or condition marked by a high level of risk or susceptibility. Risk assessment consists of identifying and characterizing the nature, frequency, and severity of the risks associated with using a product.

“Safety” means the incidence or severity of adverse events associated with the administration of an active agent, including adverse effects associated with patient-related factors (e.g., age, gender, ethnicity, race, target illness, abnormalities of renal or hepatic function, co-morbid illnesses, genetic characteristics such as metabolic status, or environment) and active agent-related factors (e.g., dose, plasma level, duration of exposure, or concomitant medication).

“Up-titration” of a compound refers to increasing the amount of a compound to achieve a therapeutic effect that occurs before dose-limiting intolerability for the patient. Up-titration can be achieved in one or more dose increments, which may be the same or different.

“Antipsychotic” refers to a drug that diminishes psychosis, hallucinations, or delusions. Antipsychotics include, but are not limited to, haloperidol, droperidol, chlorpromazine, fluphenazine, perphenazine, prochlorperazine, thioridazine, trifluoperazine, mesoridazine, periciazine, promazine, triflupromazine, levomepromazine, promethazine, pimozide, chlorprothixene, flupenthixol, thiothixene, zuclopenthixol, clozapine, olanzapine, risperidone, quetiapine, ziprasidone, amisulpride, asenapine, paliperidone, zotepine, aripiprazole, bifeprunox, and tetrabenazine.

“Anxiolytics” refers to drugs that reduce anxiety, fear, panic, or related feelings. Such drugs include, but are not limited to, benzodiazepines (e.g., alprazolam, chlordiazepoxide, clonazepam, clorazepate, diazepam, lorazepam), buspirone, barbiturates (e.g., amobarbital, pentobarbital, secobarbital, phenobarbital), and hydroxyzine.

“Antidepressant” refers to a drug that alleviates depression and related conditions (e.g., dysthymia). Such drugs include, but are not limited to, selective serotonin-reuptake inhibitors (SSRIs, e.g., citalopram, escitalopram, fluoxetine, fluvoxamine, paroxetine, sertraline), serotonin-norepinephrine reuptake inhibitors (SNRIs, e.g., desvenlafaxine, duloxetine, milnacipran, venlafaxine), mianserin, mirtazapine, norepinephrine reuptake inhibitors (e.g., atomoxetine, mazindol, reboxetine, viloxazine), bupropion, tianeptine, agomelatine, tricyclic antidepressants (e.g., amitriptyline, clomipramine, doxepin, imipramine, trimipramine, desipramine, nortriptyline, protriptyline), and monoamine oxidase inhibitors (e.g., isocarboxazid, moclobemide, phenelzine, selegiline, tranylcypromine).

“Sedatives” or “tranquilizers” refer to drugs that induce somnolence, promote a feeling of being tired or desire to sleep, or promote a state of unconsciousness. Such drugs include, but are not limited to, benzodiazepines, barbiturates (e.g., amobarbital, pentobarbital, secobarbital, phenobarbital), eszopiclone, zaleplon, zolpidem, and zopiclone.

“Mini-Mental State Examination” (MMSE) is a brief 30-point questionnaire used to quantitatively assess cognition. The MMSE includes simple questions and problems in several areas: the time and place of testing, repeating lists of words, arithmetic, language use, comprehension, and copying a drawing.

Neuropsychiatric Inventory Clinician (NPI-C) is a valid and reliable clinician-administered scale used to assess the occurrence, severity, and meaningful change in neuropsychiatric symptoms specific to those experienced by people with dementia. It has fourteen domains: delusions, hallucinations, agitation, aggression, dysphoria, anxiety, elation/euphoria, apathy/indifference, disinhibition, irritability/lability, aberrant motor disturbances, sleep disorders, appetite and eating disorders, and aberrant vocalizations.

A clinical impression rating is assigned to each NPI-C item for the specific domain. The clinician rater assigns the rating incorporating the sources of information available (caregiver interview, patient interview, patient data, other relevant information) and is focused on the previous four-week timeframe. The clinical impression is rated on 0 to 3 point scale; 0=none; 1=mild: produces little stress; 2=moderate: distressing and causes substantial behavioral abnormalities; 3=marked: a major source of behavioral abnormality.

The NPI-C Core scale includes four domains from the NPI-C scale, namely, delusions, hallucinations, agitation, and aggression. These four domains include the following number of items to be rated by the clinician: delusions, 8 items (maximum score=24); hallucinations, 7 items (maximum score=21); agitation, 13 items (maximum score=39); and aggression, 8 items (maximum score=24). The maximum score for the NPI-C Core scale is 108. In certain embodiments, the NPI-C assessment is performed before the other scale assessments at the visits at which it occurs. The four domains of NPI-C may be analyzed together independently, or in any combination thereof to assess potential patient benefits. In particular, the NPIC-C hallucinations and delusions domains are particularly relevant when assessing the potential benefits on psychosis.

### EXAMPLES

The following examples are provided for illustration and are not intended to limit the scope of the disclosure.

### Example 1—SPA GTP Binding Assay

Membranes were prepared for GTPgS binding assays from CHO-K1 cells stably expressing human M2 or human M4 receptors. Cells were grown in 5-layer cell culture flasks. Cells were washed with 50 mL of PBS and then dissociated with 0.05% trypsin. Dissociated cells were then collected by centrifugation at 650×g for 8 min at 4° C. Pellets were rewashed in PBS by centrifugation and resuspended in 30 mL of 20 mM HEPES, 10 mM EDTA, pH 7.4. After sitting on ice for 30 min, membranes were collected by centrifugation at 50000×g for 15 min at 4° C., resuspended in 30 mL of 20 mM HEPES, 0.1 mM EDTA, pH 7.4, and collected again by centrifugation. Pellets were then washed twice by centrifugation in 30 mL of 20 mM HEPES, pH 7.4, and stored at −80° C. until the day of the assay. On the day of the assay, pellets were thawed on ice and resuspended in 1 mL of 20 mM HEPES, pH 7.4, 10 mM MgCl₂, and 100 mM NaCl using Dounce homogenization.

According to Promega guidelines, membrane protein concentration was determined by BCA protein assay (Promega, Madison, WI).

On the day of the assay, serial dilutions of test PAM compounds were brought up in assay buffer (20 mM HEPES, 10 mM MgCl₂; see Table 4 for NaCl concentrations) at four times the final assay concentration. 25 mL/well of membrane homogenates (containing 5 mg of membrane protein) were prepared in assay buffer supplemented with GDP at 4× final assay concentration (see Table 4 for GDP concentrations) and dispensed into 96 well polypropylene, U-bottom Greiner plates. Membrane plates were incubated for 10 min at room temperature with gentle shaking. Then, 25 μL of test compounds were added to assay plates. Plates were then incubated, with shaking, for an additional 10-15 min. Then, 25 μL of diluted agonists serially diluted in assay buffer at 4× final assay concentration was added to assay plates. Plates were then incubated, with shaking, for an additional 10-15 min. Next, 25 mL of 0.4 nM ³⁵S-GTP (Perkin Elmer, Waltham, MA) in assay buffer was added to assay mixtures. The binding reaction was left on the shaker for 1 h at room temperature. Membranes in assay reactions were then transferred to GF/C filter plates (UniFilter, Perkin Elmer) and washed three times using FilterMate Harvester (Perkin Elmer) with cold assay buffer containing no GDP. Filter plates were then dried at 40° C. overnight, and 20 mL/well of Beta Plate Scintillation liquid was added. Plates were top and bottom sealed before being read on the TopCount scintillation counter (Perkin Elmer, Waltham, MA).

TABLE 4
Reagent concentrations employed in
GTPgS curve shift binding assays

| | Assay Buffer | Assay Buffer |
|---|---|---|
| | NaCl Concentration | GDP Concentration |
| M4 | 100 mM | 5 mM |
| M2 | 100 mM | 5 mM |

### Example 2—HTRF IP-One Curve Shift Assays (M1, M3, and M5 Receptors)

HTRF IP-One assays were performed using frozen cell aliquots prepared from CHO-K1 cell lines stably expressing human M1, M3, or M5 receptors. Cells were frozen following typical cell freezing protocols. Cells were washed with phosphate-buffered saline (PBS) and dissociated from tissue culture flasks with 0.25% trypsin. Dissociated cells were quenched in full media and collected by centrifugation at 290×g for 5 min. Cells were resuspended in cell freeze media (10% DMSO, 90% Avantor Seradigm FB Essence Cat #10803-034) to a concentration of 2×10⁷ cells/mL. 1 mL aliquots of cells were dispensed into cryovial tubes and frozen in a controlled rate cryo-freezer container at −80° C. Cells were stored at −80° C. (or in liquid nitrogen if stored for at least six months) until the day of the assay.

On the day before the assay, frozen cells were rapidly thawed in a 37° C. water bath, brought up in 20 mL of full media, and collected via centrifugation at 290×g for 5 min. Cells were washed in PBS by centrifugation and resuspended in Opti-MEM (Gibco Cat #31985070) to a concentration of 10⁶ cells/mL. Next, 10 mL/well of cells (10,000 cells/well) were dispensed into 384-well coming plates in triplicate. Cells were incubated overnight in a 37° C., 5% CO₂ cell incubator.

On the day of the assay, serial dilutions of test PAM compounds were brought up in assay buffer (20 mM TRIS, 150 mM NaCl, 175 mM LiCl) at 7× final assay concentration. Then, 2 μL of test compounds were added to the cells and incubated for 10 min at 37° C. in a 5% CO₂ cell incubator. Next, 2 μL of diluted agonists serially diluted in assay buffer at 7× final assay concentration was added to assay plates. Finally, assay plates were incubated for two hours at 37° C. in a 5% CO₂ cell incubator.

Six μL of IP-1 detection reagents (IP-One Gq HTRF kit, Cisbio Cat #62IPAPEJ) were dispensed to the cells. First, the plates were incubated for two hours at room temperature (IP-One detection reagents were made according to Cisbio protocol). Then, plates were read on an Envision plate reader following the Cisbio guidelines.

Raw data for all assays was transmitted directly from plate readers into the Beacon database for processing. All assay plates contained positive and negative control wells for data normalization and scaling. Negative control wells contained assay buffer, and positive control wells contained ten mM acetylcholine. All control wells contained DMSO at a final assay concentration that matched the test compound wells.

For GTPgS assays, the negative control wells were defined as 0% response. The highest response in a plate was scaled to 1, representing 100% response. Data from test compound wells was scaled to these controls and are reported as % control values. This was done to allow for subsequent GraphPad cooperativity analysis, setting the maximal response at no more than 1.

In HTRF IP-One assays, in addition to buffer and acetylcholine containing negative and positive control wells, all assay plates contained an 8-point, quadruplicate IP1 standard curve. Counts from the standard curve wells were used to generate a sigmoidal standard curve, which was used to transform the raw counts from each well on the plate (scaling control wells and compound wells) into a pmol/well IP1 value. The pmol/well values for compound wells were scaled to the values in the positive and negative control wells (10 M acetylcholine and buffer, respectively) to generate % control values. As with the GTP assays, reported agonist efficacies represent the % efficacy compared to a maximal acetylcholine response. As in the case of the GTPgS assay, efficacy was scaled to 1 for subsequent GraphPad analysis.

Results were graphed using GraphPad with the equation below, previously described in Black J W and Leff P (1983) “Operational models of pharmacological agonism,” Proc R Soc Lond B Biol Sci 220:141-162, incorporated herein by reference:

$$Response = \frac{{τ}_{A} [ A ] / {K}_{A} ( 1 + αβ [ B ] / {K}_{B} ) + {τ}_{B} [ B ] / {K}_{B}}{[ A ] / {K}_{A} ( 1 + α [ B ] / {K}_{B} + {τ}_{A} ( 1 + αβ [ B ] / {K}_{B} ) ) + [ B ] / {K}_{B} ( 1 + {τ}_{B} ) + 1}$$

### Example 3—Analysis of Efficacy and Potency Across Muscarinic Receptor Subtypes

Combinations of M4 orthosteric modulators and PAMs were tested across the multiple receptors to demonstrate a preferential effect for muscarinic receptor subtypes. M2 and M4 receptors were tested using the SPA GTP Binding Assay of Example 1. M1, M3, and M5 receptors were tested using the HTRF IP-one curve shift assays of Example 2.

KTX-517 refers to the C₅-xanomeline analog, 3-(1-methyl-1,4,5,6-tetrahydropyridin-2-yl)-4-(pentyloxy)-1,2,5-thiadiazole, having the structure

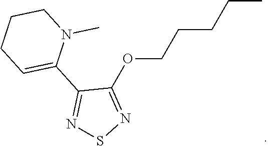 [SMILES: CCCCCOc1nsnc1C1=CCCCN1C]

KTX-593 refers to the C₇-xanomeline analog, 3-(heptyloxy)-4-(1-methyl-1,4,5,6-tetrahydropyridin-2-yl)-1,2,5-thiadiazole, having the structure S

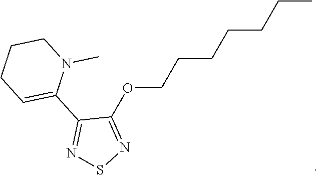 [SMILES: CCCCCCCOc1nsnc1C1=CCCCN1C]

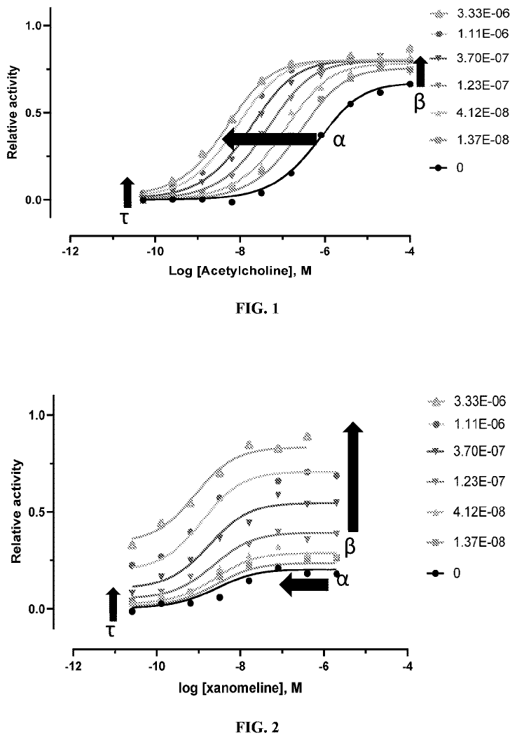

FIGS. 1 and 2 show the in vitro cooperativity of MK-4710 with acetylcholine (FIG. 1) and xanomeline (FIG. 2) at the M4 mAChR. Referring to FIG. 1, MK-4710 with acetylcholine is associated with a robust leftward shift in affinity (alpha), a modest increase in efficacy (beta), and no agonist (tau) pharmacology. Referring now to FIG. 2, MK-4710 with xanomeline is associated with a modest leftward shift in affinity (alpha), a robust increase in efficacy (beta) and moderate agonist (tau) pharmacology. These results are also summarized below in Table 5, along with the results for emraclidine.

TABLE 5
Cooperativity Table

| | | M4 PAM | | | | | |
|---|---|---|---|---|---|---|---|
| mAChR | Orthosteric | Emraclidine | | | MK-4710 | | |
| subtype | agonist | α | β | τ | α | β | τ |
| M1 | acetylcholine | 2.1 | 0.8 | 0.0 | 1.1 | 1.4 | 0.1 |
| | xanomeline | 1.2 | 1.6 | 0.0 | 1.5 | 3.8 | −0.1 |
| | cevimeline | 1.0 | 1.2 | 0.0 | 3.0 | 0.5 | 0.0 |
| | KTX-517 | 11.3 | 2.2 | 0.0 | 3.5 | 0.6 | 0.0 |
| | KTX-593 | 3.2 | 0.8 | 0.0 | 4.1 | 0.6 | 0.0 |
| M2 | acetylcholine | 3.9 | 1.4 | 0.0 | 10.5 | 2.1 | 0.0 |
| | xanomeline | 1.0 | 3.6 | 0.0 | 1.0 | 2.5 | 0.0 |
| | cevimeline | 1.0 | 21.1 | 0.0 | 1.0 | 53.4 | 0.0 |
| | KTX-517 | 1.0 | 18.1 | 0.1 | 1.0 | 5.0 | 0.1 |
| | KTX-593 | 1.0 | 1370.0 | 1.6 | 1.0 | 10.3 | 0.0 |
| M3 | acetylcholine | 1.3 | 2.6 | 0.0 | 1.7 | 1.9 | 0.0 |
| | xanomeline | 1.0 | 1.7 | 0.1 | 1.0 | 0.9 | 0.1 |
| | cevimeline | 1.0 | 1.1 | 0.1 | 2.1 | 0.6 | 0.0 |
| | KTX-517 | 1.0 | 2.8 | 0.0 | 7.7 | 1.1 | 0.0 |
| | KTX-593 | 7.6 | 1.1 | 0.0 | 6.2 | 1.6 | 0.0 |
| M4 | acetylcholine | 315.4 | 2.8 | 0.1 | 168.1 | 2.4 | 0.0 |
| | xanomeline | 1.0 | 90.4 | 1.4 | 1.0 | 993.4 | 0.5 |
| | cevimeline | 1.0 | 57.2 | 0.4 | 1.0 | 136.5 | 0.1 |
| | KTX-517 | 3.0 | 32.3 | 0.6 | 1.0 | 1395.0 | 0.8 |
| | KTX-593 | 2.4 | 56.3 | 1.5 | 1.0 | 1051018.0 | 381.2 |
| M5 | acetylcholine | 2.1 | 5.3 | 0.0 | 1.0 | 1.6 | 0.0 |
| | xanomeline | 1.0 | 1.0 | 0.1 | 1.0 | 1.3 | 0.0 |
| | cevimeline | 1.0 | 0.5 | 0.1 | 1.0 | 1.4 | 0.1 |
| | KTX-517 | 1.0 | 4.5 | 0.1 | 2.2 | 1.9 | 0.0 |
| | KTX-593 | 10.6 | 1.1 | 0.0 | 1.4 | 0.8 | 0.1 |

As shown in Tables 6-15, cooperativity was found between M4 PAMs and orthosteric synthetic mnuscarinic agonists. The cooperativity between the PAMs and agonists was unique relative to cooperativity observed with PAMs and acetylcholine, the native muscarinic receptor ligand. Predominant cooperativity was observed on beta (efficacy) when synthetic muscarinic agonists were applied with M4 PAMs, including xanomeline, cevimeline, and the C₅ and C₇ xanomeline analogs, while the predominant cooperativity of M4 PAMs with acetylcholine was on alpha (potency), obtained by SPA GTP binding assay.

Secondly, regarding subtype specificity, a nonselective muscarinic receptor agonist when combined with an M4 PAM preferentially activated the M4 mAChR but not the other mAChRs. Subtype specific was also demonstrated for the M2 receptor, as shown by SPA GTP Binding Assay, and M1, M3, and M5 as demonstrated by the IP1 assay.

Thus, the PAM/agonist combination may promote the desired effects of one receptor, e.g. M4, while downgrading the effects or leaving the remaining receptors (e.g., M1, M3, and M5) largely unaffected in manner not possible with only acetylcholine.

TABLE 6
M1 potency and efficacy table for emraclidine

| Emraclidine | w/acetylcholine | | w/xanomeline | | w/cevimeline | | w/KTX-517 | | w/KTX-593 | |
|---|---|---|---|---|---|---|---|---|---|---|
| concentration | EC₅₀ | Eₘₐₓ | EC₅₀ | Eₘₐₓ | EC₅₀ | Eₘₐₓ | EC₅₀ | Eₘₐₓ | EC₅₀ | Eₘₐₓ |
| (nM) | (nM) | (%) | (nM) | (%) | (nM) | (%) | (nM) | (%) | (nM) | (%) |
| 0 | 96.9 | 83.9 | 8.7 | 74.7 | 1194.0 | 73.6 | 7.0 | 61.6 | 10.1 | 77.8 |
| 13.7 | 51.3 | 71.8 | 8.5 | 75.6 | 1202.0 | 73.2 | 6.9 | 61.0 | 11.0 | 83.9 |
| 41.2 | 61.6 | 76.7 | 10.4 | 79.9 | 1340.0 | 81.4 | 7.1 | 65.6 | 6.9 | 83.1 |
| 123 | 57.2 | 79.9 | 13.5 | 83.5 | 1362.0 | 90.4 | 6.5 | 66.5 | 7.4 | 85.4 |
| 370 | 55.0 | 78.3 | 11.2 | 87.3 | 1748.0 | 95.1 | 5.8 | 71.3 | 5.3 | 88.4 |
| 1110 | 66.2 | 79.5 | 9.5 | 90.0 | 2029.0 | 100.2 | 5.9 | 72.8 | 4.7 | 85.2 |
| 3330 | 68.6 | 73.4 | 8.6 | 87.9 | 2010.0 | 103.1 | 8.9 | 79.0 | 6.5 | 87.8 |

TABLE 7
M1 potency and efficacy table for MK-4710

| MK-4710 | w/acetylcholine | | w/xanomeline | | w/cevimeline | | w/KTX-517 | | w/KTX-593 | |
|---|---|---|---|---|---|---|---|---|---|---|
| concentration | EC₅₀ | Eₘₐₓ | EC₅₀ | Eₘₐₓ | EC₅₀ | Eₘₐₓ | EC₅₀ | Eₘₐₓ | EC₅₀ | Eₘₐₓ |
| (nM) | (nM) | (%) | (nM) | (%) | (nM) | (%) | (nM) | (%) | (nM) | (%) |
| 0 | 136.1 | 79.1 | 7.6 | 78.2 | 1754.0 | 86.1 | 8.4 | 80.9 | 8.7 | 83.7 |
| 13.7 | 76.1 | 86.1 | 10.9 | 85.6 | 1704.0 | 88.8 | 8.7 | 77.8 | 8.8 | 77.6 |
| 41.2 | 83.9 | 86.9 | 10.9 | 87.8 | 2034.0 | 102.4 | 8.5 | 81.5 | 7.6 | 82.7 |
| 123 | 74.8 | 90.6 | 12.8 | 88.1 | 2087.0 | 97.1 | 10.4 | 79.6 | 8.3 | 79.9 |
| 370 | 79.9 | 85.8 | 14.1 | 85.9 | 1917.0 | 92.3 | 7.5 | 80.9 | 6.5 | 78.3 |
| 1110 | 69.8 | 84.0 | 14.7 | 81.4 | 1842.0 | 83.6 | 7.7 | 86.1 | 6.8 | 76.8 |
| 3330 | 80.2 | 82.5 | 11.7 | 78.4 | 1717.0 | 69.9 | 7.4 | 85.8 | 5.3 | 76.7 |

TABLE 8
M2 potency and efficacy table for emraclidine

| Emraclidine | w/acetylcholine | | w/xanomeline | | w/cevimeline | | w/KTX-517 | | w/KTX-593 | |
|---|---|---|---|---|---|---|---|---|---|---|
| concentration | EC₅₀ | Eₘₐₓ | EC₅₀ | Eₘₐₓ | EC₅₀ | Eₘₐₓ | EC₅₀ | Eₘₐₓ | EC₅₀ | Eₘₐₓ |
| (nM) | (nM) | (%) | (nM) | (%) | (nM) | (%) | (nM) | (%) | (nM) | (%) |
| 0 | 1487.0 | 71.4 | 41.1 | 36.2 | 2100.0 | 34.0 | 18.5 | 35.6 | 35.7 | 39.8 |
| 13.7 | 1200.0 | 77.1 | 24.6 | 41.7 | 2882.0 | 43.8 | 13.9 | 44.8 | 55.1 | 48.7 |
| 41.2 | 1320.0 | 82.0 | 42.4 | 48.2 | 2999.0 | 57.0 | 17.4 | 57.9 | 30.8 | 52.6 |
| 123 | 1628.0 | 83.7 | 36.4 | 49.8 | 2343.0 | 51.8 | 7.4 | 51.1 | 22.8 | 52.2 |
| 370 | 1374.0 | 82.4 | 24.0 | 41.5 | 2460.0 | 48.6 | 5.7 | 52.6 | 55.7 | 50.1 |
| 1110 | 651.1 | 79.7 | 33.2 | 59.6 | 2259.0 | 51.4 | 7.0 | 65.9 | 28.1 | 67.5 |
| 3330 | 751.2 | 90.5 | 35.2 | 64.1 | 1654.0 | 69.6 | 7.1 | 79.2 | 14.9 | 75.1 |

TABLE 9
M2 potency and efficacy table for MK-4710

| MK-4710 | w/acetylcholine | | w/xanomeline | | w/cevimeline | | w/KTX-517 | | w/KTX-593 | |
|---|---|---|---|---|---|---|---|---|---|---|
| concentration | EC₅₀ | Eₘₐₓ | EC₅₀ | Eₘₐₓ | EC₅₀ | Eₘₐₓ | EC₅₀ | Eₘₐₓ | EC₅₀ | Eₘₐₓ |
| (nM) | (nM) | (%) | (nM) | (%) | (nM) | (%) | (nM) | (%) | (nM) | (%) |
| 0 | 1535.0 | 79.0 | 28.5 | 55.6 | 2264.0 | 39.6 | 14.3 | 45.1 | 19.6 | 38.7 |
| 13.7 | 1588.0 | 84.7 | 22.8 | 60.1 | 3885.0 | 42.1 | 25.7 | 60.6 | 102.5 | 57.0 |
| 41.2 | 675.0 | 74.6 | 31.4 | 65.3 | 1250.0 | 38.2 | 11.9 | 53.2 | 60.0 | 50.1 |
| 123 | 653.4 | 85.9 | 47.2 | 74.7 | 1423.0 | 39.0 | 14.8 | 55.6 | 29.3 | 50.2 |
| 370 | 435.0 | 89.3 | 38.4 | 55.5 | 1325.0 | 39.3 | 13.1 | 59.1 | 37.4 | 61.4 |
| 1110 | 187.7 | 85.0 | 51.6 | 74.1 | 1557.0 | 49.5 | 15.8 | 70.6 | 23.4 | 62.2 |
| 3330 | 97.7 | 84.8 | 37.9 | 75.7 | 1243.0 | 68.2 | 10.3 | 78.7 | 28.3 | 77.6 |

TABLE 10
M3 potency and efficacy table for emraclidine

| Emraclidine | w/acetylcholine | | w/xanomeline | | w/cevimeline | | w/KTX-517 | | w/KTX-593 | |
|---|---|---|---|---|---|---|---|---|---|---|
| concentration | EC₅₀ | Eₘₐₓ | EC₅₀ | Eₘₐₓ | EC₅₀ | Eₘₐₓ | EC₅₀ | Eₘₐₓ | EC₅₀ | Eₘₐₓ |
| (nM) | (nM) | (%) | (nM) | (%) | (nM) | (%) | (nM) | (%) | (nM) | (%) |
| 0 | 86.2 | 72.1 | 155.5 | 77.3 | 3269.0 | 97.9 | 24.4 | 42.7 | 223.6 | 63.5 |
| 13.7 | 61.2 | 68.0 | 120.8 | 74.5 | 2149.0 | 77.3 | 32.3 | 43.0 | 310.9 | 71.1 |
| 41.2 | 85.9 | 74.2 | 156.0 | 81.9 | 3613.0 | 96.7 | 22.7 | 45.3 | 97.4 | 63.9 |
| 123 | 83.1 | 77.6 | 157.3 | 85.7 | 2163.0 | 83.8 | 42.8 | 50.2 | 104.0 | 68.2 |
| 370 | 79.6 | 77.1 | 211.7 | 91.6 | 2181.0 | 87.8 | 27.4 | 52.1 | 153.0 | 73.4 |
| 1110 | 94.9 | 82.4 | 149.7 | 89.1 | 5000.0 | 125.3 | 18.9 | 54.8 | 46.7 | 64.6 |
| 3330 | 78.0 | 76.0 | 123.3 | 80.3 | 2266.0 | 83.9 | 30.1 | 59.6 | 93.1 | 73.0 |

TABLE 11
M3 potency and efficacy table for MK-4710

| MK-4710 | w/acetylcholine | | w/xanomeline | | w/cevimeline | | w/KTX-517 | | w/KTX-593 | |
|---|---|---|---|---|---|---|---|---|---|---|
| concentration | EC₅₀ | Eₘₐₓ | EC₅₀ | Eₘₐₓ | EC₅₀ | Eₘₐₓ | EC₅₀ | Eₘₐₓ | EC₅₀ | Eₘₐₓ |
| (nM) | (nM) | (%) | (nM) | (%) | (nM) | (%) | (nM) | (%) | (nM) | (%) |
| 0 | 105.2 | 67.5 | 146.8 | 84.2 | 6365.0 | 118.7 | 33.8 | 46.7 | 45.0 | 80.8 |
| 13.7 | 71.6 | 77.2 | 180.0 | 81.5 | 11500.0 | 158.6 | 26.6 | 45.6 | 47.2 | 72.7 |
| 41.2 | 66.3 | 84.3 | 185.7 | 94.3 | 7600.0 | 150.7 | 24.4 | 49.5 | 35.6 | 75.4 |
| 123 | 70.3 | 188.2 | 211.1 | 90.3 | 4876.0 | 136.9 | 25.6 | 48.3 | 34.1 | 66.6 |
| 370 | 75.5 | 184.7 | 227.0 | 88.9 | 9674.0 | 162.9 | 22.7 | 53.4 | 34.1 | 76.7 |
| 1110 | 73.1 | 85.4 | 220.1 | 81.0 | 11160.0 | 149.7 | 29.6 | 51.1 | 32.0 | 74.3 |
| 3330 | 80.1 | 84.8 | 208.4 | 81.8 | 4107.0 | 93.1 | 23.3 | 64.4 | 58.3 | 86.0 |

TABLE 12
M4 potency and efficacy table for emraclidine

| Emraclidine | w/acetylcholine | | w/xanomeline | | w/cevimeline | | w/KTX-517 | | w/KTX-593 | |
|---|---|---|---|---|---|---|---|---|---|---|
| concentration | EC₅₀ | Eₘₐₓ | EC₅₀ | Eₘₐₓ | EC₅₀ | Eₘₐₓ | EC₅₀ | Eₘₐₓ | EC₅₀ | Eₘₐₓ |
| (nM) | (nM) | (%) | (nM) | (%) | (nM) | (%) | (nM) | (%) | (nM) | (%) |
| 0 | 797.9 | 76.8 | 11.4 | 27.2 | 1300.0 | 4.0 | 1.6 | 2.6 | 12.7 | 14.3 |
| 13.7 | 390.5 | 84.2 | 3.8 | 28.7 | 1401.0 | 10.6 | 2.0 | 10.6 | 5.3 | 24.2 |
| 41.2 | 159.0 | 83.7 | 3.1 | 31.9 | 270.1 | 13.2 | 5.8 | 17.8 | 2.8 | 24.5 |
| 123 | 69.8 | 86.2 | 1.3 | 38.1 | 746.4 | 28.0 | 1.7 | 29.6 | 1.0 | 43.9 |
| 370 | 22.5 | 84.1 | 3.7 | 60.0 | 500.4 | 49.2 | 1.4 | 46.5 | 0.8 | 60.1 |
| 1110 | 9.1 | 86.7 | 0.9 | 78.7 | 298.4 | 73.3 | 1.8 | 62.4 | 1.4 | 76.8 |
| 3330 | 3.9 | 83.2 | 0.5 | 89.4 | 197.5 | 86.3 | 0.6 | 66.8 | 0.4 | 84.0 |

TABLE 13
M4 potency and efficacy table for MK-4710

| MK-4710 | w/acetylcholine | | w/xanomeline | | w/cevimeline | | w/KTX-517 | | w/KTX-593 | |
|---|---|---|---|---|---|---|---|---|---|---|
| concentration | EC₅₀ | Eₘₐₓ | EC₅₀ | Eₘₐₓ | EC₅₀ | Eₘₐₓ | EC₅₀ | Eₘₐₓ | EC₅₀ | Eₘₐₓ |
| (nM) | (nM) | (%) | (nM) | (%) | (nM) | (%) | (nM) | (%) | (nM) | (%) |
| 0 | 640.1 | 67.6 | 5.8 | 19.7 | 3015.0 | 6.4 | 547.9 | 12.6 | 44.7 | 14.0 |
| 13.7 | 191.2 | 76.8 | 1.9 | 25.1 | 2173.0 | 14.4 | 8.7 | 20.4 | 2.9 | 26.0 |
| 41.2 | 113.5 | 81.8 | 1.7 | 29.7 | 1086.0 | 20.2 | 2.3 | 27.3 | 10.1 | 35.9 |
| 123 | 37.0 | 78.1 | 3.3 | 38.5 | 813.6 | 26.6 | 1.4 | 34.3 | 0.8 | 41.8 |
| 370 | 20.2 | 80.8 | 2.0 | 55.0 | 473.2 | 42.1 | 2.1 | 53.4 | 1.0 | 59.7 |
| 1110 | 8.4 | 80.5 | 1.0 | 70.6 | 365.4 | 68.1 | 2.3 | 75.1 | 1.5 | 80.0 |
| 3330 | 4.2 | 81.6 | 0.6 | 80.5 | 306.3 | 83.6 | 1.5 | 87.1 | 0.6 | 88.1 |

TABLE 14
M5 potency and efficacy table for emraclidine

| Emraclidine | w/acetylcholine | | w/xanomeline | | w/cevimeline | | w/KTX-517 | | w/KTX-593 | |
|---|---|---|---|---|---|---|---|---|---|---|
| concentration | EC₅₀ | Eₘₐₓ | EC₅₀ | Eₘₐₓ | EC₅₀ | Eₘₐₓ | EC₅₀ | Eₘₐₓ | EC₅₀ | Eₘₐₓ |
| (nM) | (nM) | (%) | (nM) | (%) | (nM) | (%) | (nM) | (%) | (nM) | (%) |
| 0 | 142.3 | 84.4 | 253.2 | 81.14 | 1369 | 90.54 | 121.4 | 41.68 | 403.2 | 63.6 |
| 13.7 | 94.5 | 72.3 | 248.4 | 79.38 | 2780 | 102.1 | 110.9 | 44.07 | 376.8 | 67.08 |
| 41.2 | 118.4 | 77.7 | 278.9 | 78.5 | 2623 | 92.71 | 110.3 | 45.38 | 475.5 | 71.77 |
| 123 | 130.3 | 80.3 | 281.6 | 84.17 | 2794 | 93.45 | 117.8 | 62.69 | 346.4 | 68.82 |
| 370 | 144.0 | 82.7 | 329.9 | 89.04 | 4154 | 104.6 | 160.7 | 62.4 | 205.2 | 67.43 |
| 1110 | 170.8 | 89.9 | 282.3 | 81.91 | 3481 | 102.7 | 242.9 | 69.34 | 236.4 | 66.87 |
| 3330 | 122.4 | 81.4 | 242.5 | 79.5 | 3769 | 106.4 | 152.8 | 72.04 | 314 | 71.88 |

TABLE 15
M5 potency and efficacy table for MK-4710

| MK-4710 | w/acetylcholine | | w/xanomeline | | w/cevimeline | | w/KTX-517 | | w/KTX-593 | |
|---|---|---|---|---|---|---|---|---|---|---|
| concentration | EC₅₀ | Eₘₐₓ | EC₅₀ | Eₘₐₓ | EC₅₀ | Eₘₐₓ | EC₅₀ | Eₘₐₓ | EC₅₀ | Eₘₐₓ |
| (nM) | (nM) | (%) | (nM) | (%) | (nM) | (%) | (nM) | (%) | (nM) | (%) |
| 0 | 242.9 | 71.2 | 351.3 | 77.91 | 3722 | 70.31 | 167.8 | 53.95 | 244.6 | 81.82 |
| 13.7 | 204.9 | 77.7 | 319 | 79.99 | 5092 | 97.31 | 214.9 | 54.93 | 188.8 | 69.69 |
| 41.2 | 297.8 | 88.9 | 323.1 | 87.05 | 3929 | 95.2 | 166.1 | 62.51 | 160.9 | 78.32 |
| 123 | 200.5 | 85.7 | 339.3 | 86.91 | 2485 | 88.22 | 181 | 65.68 | 208.2 | 78.75 |
| 370 | 231.0 | 85.9 | 429.1 | 92.87 | 4274 | 91.07 | 71.74 | 67.98 | 206 | 76.59 |
| 1110 | 214.8 | 78.1 | 340.4 | 78.6 | 3795 | 78.35 | 100.4 | 69.59 | 165.5 | 69.65 |
| 3330 | 211.5 | 78.8 | 393.7 | 82.95 | 5078 | 91.21 | 132.4 | 82.24 | 196.7 | 77.27 |

The preceding description is given for clearness of understanding. No unnecessary limitations should be understood from there, as modifications within the disclosure scope may be apparent to those having ordinary skill in the art. Throughout the specification, where compositions are described as including components or materials, it is contemplated that the compositions can also consist essentially of, or consist of, any combination of the recited components or materials, unless described otherwise. Likewise, where methods are described as including steps, it is contemplated that the methods can also consist essentially of, or consist of, any combination of the recited steps, unless described otherwise. The disclosure illustratively disclosed herein suitably may be practiced in the absence of any element or step not specifically disclosed herein.

The practice of a method disclosed herein, and individual steps thereof, can be performed manually and/or with automation provided by electronic equipment. Although processes have been described regarding embodiments, a person of ordinary skill in the art will readily appreciate that other ways of performing the acts associated with the methods may be used. For example, the order of various steps may be changed without departing from the scope or spirit of the method unless described otherwise. Some individual steps can also be combined, omitted, or further subdivided into additional steps.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination. All combinations of the embodiments of the chemical groups represented by the variables contained within the generic chemical formulae described herein are specifically embraced by the present invention just as if each combination was individually explicitly recited, to the extent that such combinations embrace stable compounds (i.e., compounds that can be isolated, characterized and tested for biological activity). Also, all subcombinations of the chemical groups listed in the embodiments describing such variables, as well as all subcombinations of uses and medical indications described herein, are also specifically embraced by the present invention, just as if each subcombination of chemical groups and subcombination of uses and medical indications was individually and explicitly recited herein.

All patents, publications, and references cited herein are hereby incorporated by reference. In case of conflict between the present disclosure and incorporated patents, publications, and references, the present disclosure should control.

1. A method for treating a disorder ameliorated by muscarinic receptor activation in a patient in need thereof, comprising administering to the patient a muscarinic orthosteric agonist and an M1 or M4 muscarinic positive allosteric modulator (PAM).

2. The method of claim 1, wherein the PAM is an M1 PAM selected from the group consisting of BQCA, GSK 1034702, M1-PAM-A, M1-PAM-B, MK-7622, PF-6767832, PQCA, TAK-071, VU319, VU0119498, VU0453595, VU0456940, VU0486846, VU0550164, VU6004256, VU6005877, and VU6007477.

3. The method of claim 1, wherein the PAM is an M4 PAM selected from the group consisting of emraclidine, LY2033298, LY2119620, VU0152099, VU0152100, VU0467154, VU0467485, VU0473619, VU0476406, VU6000918, VU6002703, VU6009003, VU6009453, MK-4710, and 6-(2-methyl-3-oxoisoindolin-5-yl)-5-(1-((1-methylcyclopentyl)methyl)-1H-pyrazol-4-yl)picolinonitrile.

4. The method of claim 1, wherein the muscarinic orthosteric agonist is acetylcholine and the PAM is an M4 PAM, and wherein the M4 PAM cooperates with acetylcholine through an affinity shift.

5. The method of claim 1, wherein the muscarinic orthosteric agonist is xanomeline or a salt thereof and the PAM is an M4 PAM, and wherein the M4 PAM cooperates with xanomeline through an efficacy shift.

6. The method of claim 1, wherein the muscarinic orthosteric agonist is cevimeline and the PAM is an M4 PAM, and wherein the M4 PAM cooperates with cevimeline through an efficacy shift.

7. The method of claim 1, wherein the muscarinic orthosteric agonist is 3-(1-methyl-1,4,5,6-tetrahydropyridin-2-yl)-4-(pentyloxy)-1,2,5-thiadiazole (C₅-xanomeline analog) and the PAM is an M4 PAM, and wherein the M4 PAM cooperates with C₅-xanomeline analog through an efficacy shift.

8. The method of claim 1, wherein the muscarinic orthosteric agonist is 3-(heptyloxy)-4-(1-methyl-1,4,5,6-tetrahydropyridin-2-yl)-1,2,5-thiadiazole (C₇-xanomeline analog) and the PAM is an M4 PAM, and wherein the M4 PAM cooperates with C₇-xanomeline analog through an efficacy shift.

9. The method of claim 1, wherein the disorder is chosen from dementia-related psychosis, schizophrenia, Alzheimer's disease, Parkinson's disease, depression, movement disorders, pain, drug addiction, tauopathy, and synucleinopathy.

10. The method of claim 9, wherein the dementia-related psychosis is due to Alzheimer's disease, Lewy body dementia, vascular dementia, dementia-related to Parkinson's disease, frontotemporal dementia, or another form of dementia.

11. The method of claim 1, wherein the administration is oral.

12. The method of claim 1, wherein the patient is treated for at least 7 days.

13. The method of claim 5, wherein the muscarinic orthosteric agonist is xanomeline tartrate.

14. The method of claim 1, wherein the muscarinic orthosteric agonist and the PAM are in the same pharmaceutical composition.

15. The method of claim 1, wherein the muscarinic orthosteric agonist or the PAM or both are co-administered in a reduced amount compared to the muscarinic orthosteric agonist or the PAM used alone.

16. The method of claim 1, wherein the muscarinic orthosteric agonist is in a first pharmaceutical composition and the PAM is in a second pharmaceutical composition.

17. The method of claim 16, wherein the first composition or the second composition or both are co-administered in a reduced amount compared to the first composition or the second composition used alone.

18. The method of claim 1, wherein the muscarinic orthosteric agonist is an M1 or M4 muscarinic orthosteric agonist.

19. The method of claim 1, wherein the muscarinic orthosteric agonist is xanomeline or a salt thereof.

20. The method of claim 1, further comprising administering a muscarinic antagonist.

21. The method of claim 20, wherein the muscarinic antagonist is selected from the group consisting of trospium, tolterodine, darifenacin, solifenacin, fesoterodine, scopolamine, N-methylscopolamine, and a salt thereof.

22. The method of claim 21, wherein the muscarinic antagonist is trospium chloride.

23. The method of claim 20, wherein the muscarinic antagonist and the muscarinic orthosteric agonist are administered in a first composition and the PAM is administered in a second composition.

24. A pharmaceutical composition, comprising xanomeline or a salt thereof, an M1 or M4 muscarinic positive allosteric modulator (PAM), and at least one pharmaceutically acceptable excipient.

25. The pharmaceutical composition of claim 24, wherein the excipient is chosen from microcrystalline cellulose, lactose, and talc.

26. The pharmaceutical composition of claim 24, which is formulated for oral administration.

27. The pharmaceutical composition of claim 24, which is in the form of a capsule.

28. The pharmaceutical composition of claim 24, wherein the muscarinic orthosteric agonist is an M1 or M4 muscarinic orthosteric agonist.

29. The pharmaceutical composition of claim 24, wherein the muscarinic orthosteric agonist is xanomeline or a salt thereof.

30. The pharmaceutical composition of claim 24, further comprising a muscarinic antagonist.

31. The pharmaceutical composition of claim 30, wherein the muscarinic antagonist is selected from the group consisting of trospium, tolterodine, darifenacin, solifenacin, fesoterodine, scopolamine, N-methylscopolamine, and a salt thereof.

32. The pharmaceutical composition of claim 31, wherein the muscarinic antagonist is trospium chloride.

33. The pharmaceutical composition of claim 30, wherein the muscarinic antagonist and the muscarinic orthosteric agonist are administered in a first composition and the PAM is administered in a second composition.
